(19) United States
(12) Patent Application Publication
(10) Pub. No.: US 2005/0074506 A1
(43) Pub. Date: Apr. 7, 2005

(54) Targeted release of nitric oxide in the CNS circulation for modulating the BBB and treating disorders

(75) Inventors: Amir Natan, Tel Aviv (IL); Alon Shalev, Ra'anana (IL)

Correspondence Address: DARBY & DARBY P.C., P. O. BOX 5257, NEW YORK, NY, 10150-5257, US

(73) Assignee: BRAINSGATE LTD., Ra'anana (IL)

(21) Appl. No.: 10/678,730

(22) Filed: Oct. 2, 2003

(51) Int. Cl. A61K 33/00; A61K 31/21; A61N 1/30

(52) U.S. Cl. 424/718; 514/509; 604/20

(57) ABSTRACT

A method for delivering molecules to a central nervous system (CNS) of a subject includes supplying the molecules to a blood circulation of the CNS; supplying, to a body of the subject, a carrier system that encapsulates a nitric oxide (NO) facilitator; and applying energy to the carrier system at an energy level sufficient to cause the carrier system to release the NO facilitator in a blood circulation of the subject in a vicinity of a blood-brain barrier (BBB) of the subject and thereby increase passage of the molecules from the blood circulation of the CNS, through the BBB, and into the CNS of the subject.

### FIELD OF THE INVENTION

The present invention relates generally to medical procedures, pharmaceutical compounds, and electronic devices. More specifically, the invention relates to medical procedures, pharmaceutical compounds, and electronic devices for the treatment and/or diagnosis of a clinical condition.

### BACKGROUND OF THE INVENTION

The blood-brain barrier (BBB) is a unique feature of the central nervous system (CNS) which isolates the brain and the spinal cord from the systemic blood circulation. To maintain the homeostasis of the CNS, the BBB prevents access to the CNS of many substances circulating in the blood.

The BBB is formed by a complex cellular system of endothelial cells, astroglia, pericytes, perivascular macrophages, and a basal lamina. Compared to other tissues, brain endothelia have the most intimate cell-to-cell connections: endothelial cells adhere strongly to each other, forming structures specific to the CNS called “tight junctions” or zonula occludens. They involve two opposing plasma membranes which form a membrane fusion with cytoplasmic densities on either side. These tight junctions prevent cell migration or cell movement between endothelial cells. A continuous uniform basement membrane surrounds the brain capillaries. This basal lamina encloses contractile cells called pericytes, which form an intermittent layer and probably play some role in phagocytosis activity and defense if the BBB is breached. Astrocytic end feet, which cover the brain capillaries, build a continuous sleeve and maintain the integrity of the BBB by the synthesis and secretion of soluble growth factors (e.g., gamma-glutamyl transpeptidase) essential for the endothelial cells to develop their BBB characteristics.

Because of the BBB, certain non-surgical treatments of the CNS based upon systemic introduction of compounds through the bloodstream have been ineffective or less effective. For example, chemotherapy has been relatively ineffective in the treatment of CNS metastases of systemic cancers (e.g., breast cancer, small cell lung cancer, lymphoma, and germ cell tumors), despite clinical regression and even complete remission of these tumors in non-CNS systemic locations. The most important factors determining drug delivery from blood into the CNS are lipid solubility, molecular mass, and electrical charge. A good correlation exists between the lipid solubility of a drug, expressed as the octanol/water partition coefficient, and the drug's ability to penetrate or diffuse across the BBB. This is particularly relevant for drugs with molecular weights smaller than 600 dalton (Da). The normal BBB prevents the passage of ionized water soluble drugs with a molecular weight greater than 180 Da. Most currently-available effective chemotherapeutic agents, however, have a molecular weight between 200 and 1200 Da. Therefore, based both on their lipid solubilities and molecular masses, the passage of many agents is impeded by the BBB.

In addition to transcellular diffusion of lipophilic agents, there are several specific transport mechanisms to carry certain molecules across the brain's endothelial cells. Specific transport proteins exist for required molecules, such as glucose and amino acids. Additionally, absorptive endocytosis and transcytosis occur for cationized plasma proteins. Specific receptors for certain proteins, such as transferrin and insulin, mediate endocytosis and transport across the cell.

Non-surgical treatment of neurological disorders is generally limited to systemic introduction of compounds such as neuropharmaceuticals and other neurologically-active agents that might remedy or modify neurologically-related activities and disorders. Such treatment is limited, however, by the relatively small number of known compounds that pass through the BBB. Even those that do cross the BBB often produce adverse reactions in other parts of the body or in non-targeted regions of the CNS.

There have been a number of different studies regarding efforts to cross the BBB—specifically, with regard to overcoming the limited access of drugs to the CNS. Such efforts have included, for example, chemical modification, development of more hydrophobic analogs, or linking an active compound to a specific carrier. Transient opening of the BBB in humans has been achieved by intracarotid infusion of hypertonic mannitol solutions or bradykinin analogs. Also, modulation of the P-glycoprotein, whose substrates are actively pumped out of brain cells into capillary lumens, has been found to facilitate the delivery of drugs to the brain.

PCT Publication WO 01/85094 to Shalev at al., which is assigned to the assignee of the present application and is incorporated herein by reference, describes apparatus for modifying a property of a brain of a patient. The apparatus includes one or more electrodes adapted to be applied to a site selected from a group of sites consisting of: a sphenopalatine ganglion (SPG) and a neural tract originating in or leading to the SPG. A control unit is adapted to drive the one or more electrodes to apply a current to the site capable of inducing (a) an increase in permeability of a BBB of the patient, (b) a change in cerebral blood flow of the patient, and/or (c) an inhibition of parasympathetic activity of the SPG.

U.S. Pat. No. 5,752,515 to Jolesz et al., which is incorporated herein by reference, describes apparatus for image-guided ultrasound delivery of compounds through the BBB. Ultrasound is applied to a site in the brain to effect in the tissues and/or fluids at that location a change detectable by imaging. At least a portion of the brain in the vicinity of the selected location is imaged, e.g., via magnetic resonance imaging, to confirm the location of that change. A compound, e.g., a neuropharmaceutical, in the patient's bloodstream is delivered to the confirmed location by applying ultrasound to effect opening of the BBB at that location and, thereby, to induce uptake of the compound there.

U.S. Pat. No. 6,514,221 to Hynynen et al., which is incorporated herein by reference, describes a method for opening a blood-organ barrier of a subject, including providing an exogenous agent configured to facilitate opening of the blood-organ barrier, administering the exogenous agent to a desired region of the subject, and applying energy to the desired region of the subject while the exogenous agent is present in the desired region, the energy being in a blood-organ-barrier-opening amount sufficient to induce opening of the blood-organ barrier of the subject with the exogenous agent present and below a damage amount sufficient to induce thermal damage to tissue in the absence of the exogenous agent.

U.S. Pat. No. 6,312,686 to Staddon et al., which is incorporated herein by reference, describes a method for modulating the BBB by administering an agent which promotes tyrosine protein dephosphorylation of at least one component of a cadherin/catenin complex in adherens junctions and/or tight junctions of the BBB.

U.S. Pat. No. 5,434,137 to Black, which is incorporated herein by reference, describes a method for selectively opening abnormal brain tissue capillaries of a mammal in order to allow selective passage of both low and high molecular weight neuropharmaceutical agents into abnormal brain tissue. The method utilizes direct infusion of bradykinin into the carotid artery. The dose of bradykinin is maintained at levels which provide opening of abnormal brain tissue capillaries without opening normal brain capillaries.

U.S. Pat. No. 5,686,416 to Kozarich et al., which is incorporated herein by reference, describes peptides called receptor mediated permeabilizers (RMP), which increase the permeability of the BBB to molecules such as therapeutic agents or diagnostic agents. The permeabilizer A-7 or conformational analogues can be intravenously co-administered to a host together with molecules whose desired destination is the interstitial fluid compartment of the brain.

U.S. Pat. No. 5,260,308 to Poduslo et al., which is incorporated herein by reference, describes a method for enhancing the permeability of the blood-nerve barrier (BNB) or the BBB to neuroactive proteins, comprising glycating the proteins prior to bringing them into contact with the barriers.

U.S. Pat. Nos. 5,604,198 and 5,670,477 to Poduslo et al., which are incorporated herein by reference, describe methods for enhancing the ability of a neurologically-active compound to penetrate the BNB or BBB, comprising administrating a conjugate comprising the neurologically active compound linked to a carrier molecule that has been shown to have a substantial permeability coefficient across the BNB and BBB.

U.S. Pat. No. 5,731,303 to Hsieh, which is incorporated herein by reference, describes a method for enhancing the rate of absorption of drugs across skin and other body membranes such as mucous membranes and the BBB, comprising adding to the drug composition a compound which enhances the rate. This compound may be a macrocyclic ester, diester, amide, diamide, amidine, diamidine, thioester, dithioester, thioamide, ketone, or lactone.

PCT Publication WO 00/09073 to Ekwuribe et al., which is incorporated herein by reference, describes amphiphilic drug-oligomer conjugates capable of traversing the BBB and methods of making and using such conjugates. Amphiphilic drug-oligomer conjugates comprise a therapeutic compound conjugated to an oligomer, wherein the oligomer comprises a lipophilic moiety coupled to a hydrophilic moiety. The conjugates of the invention further comprise therapeutic agents such as proteins, peptides, nucleosides, nucleotides, antiviral agents, antineoplastic agents, antibiotics, etc., and prodrugs, precursors, derivatives and intermediates thereof, chemically coupled to amphiphilic oligomers.

U.S. Pat. No. 6,436,437 to Yatvin et al., which is incorporated herein by reference, describes a method of facilitating the entry of drugs into cells and tissues at physiologically protected sites at pharmacokinetically useful levels and also a method of targeting drugs to specific organelles within the cell. The method comprises administering a compound comprising an agent for the treatment of ischemia or other vascular disease of the CNS, and a polar lipid carrier, two linker functional groups and a spacer, wherein the spacer has a first end and a second end, and wherein the polar lipid is attached to the first end of the spacer through a first linker functional group and the compound is attached to the second end of the spacer through a second linker functional group.

PCT Publication WO 03/039677 to Yatvin et al., which is incorporated herein by reference, describes a method of facilitating the entry of drugs into cells and tissues at physiologically protected sites at pharmacokinetically useful levels and also a method of targeting drugs to physiologically protected sites in vivo. The method comprises administering a compound comprising a drug conjugated with an amino acid or derivative thereof for facilitating such targeted drug delivery.

PCT Publication WO 98/22092 to Pardridge et al., which is incorporated herein by reference, describes a brain-specific liposome targeting vehicle for transporting neuropharmaceutical agents across the BBB. The targeting vehicle includes a liposome which is sterically stabilized by attaching ligands to the surface of the liposome. The targeting vehicle further includes blood-barrier transport agents which are attached to the tail portion of the stabilizing ligands which extend outward from the liposome surface. The blood-barrier transport agents are capable of transporting the entire liposome targeting vehicle across the BBB. Monoclonal antibodies which undergo receptor-mediated transcytosis across the BBB are described as useful blood-barrier transport agents.

PCT Publication WO 03/009815 to Beliveau et al., which is incorporated herein by reference, describes conjugates of therapeutic or active agents with melanotransferrin or with other ligands of a melanotransferrin receptor, melanotransferrin receptor modulators, and related compositions and methods for modulating BBB transport by providing methods of screening and selecting such conjugates, ligands, and modulators in vitro and in vivo, and methods of use of such conjugates, modulators and ligands in diagnosis and the treatment of diseases, including particularly diseases of the CNS or lysosomal storage diseases.

US Patent Application Publication 2002/0127198 to Rothbard et al., which is incorporated herein by reference, describes compositions and methods for enhancing delivery of drugs and other agents across epithelial tissues and endothelial tissues, including the BBB. The compositions and methods employ a delivery enhancing transporter that has sufficient guanidino or amidino sidechain moieties to enhance delivery of a compound conjugated to the reagent across one or more layers of the tissue, compared to the non-conjugated compound. The delivery-enhancing polymers include poly-arginine molecules that are preferably between about 6 and 25 residues in length.

The following references, which are incorporated herein by reference, may be of interest:
Delepine L, Aubineau P, “Plasma protein extravasation induced in the rat dura mater by stimulation of the parasympathetic sphenopalatine ganglion,” Experimental Neurology 147:389-400 (1997)
Hara H, Zhang Q J, Kuroyanagi T, Kobayashi S, “Parasympathetic cerebrovascular innervation: An anterograde tracing from the sphenopalatine ganglion in the rat,” Neurosurgery 32:822-827 (1993)
Jolliet-Riant P, Tillement JP, “Drug transfer across the blood-brain barrier and improvement of brain delivery,” Fundam Clin Pharmacol 13:16-25 (1999)
Kroll R A, Neuwelt E A, “Outwitting the blood brain barrier for therapeutic purposes: Osmotic opening and other means,” Neurosurgery 42:1083-1100 (1998)
Sanders M, Zuurmond W W, “Efficacy of sphenopalatine ganglion blockade in 66 patients suffering from cluster headache: A 12-70 month follow-up evaluation,” Journal of Neurosurgery 87:876-880 (1997)
Syelaz J, Hara H, Pinard E, Mraovitch S, MacKenzie E T, Edvinsson L, “Effects of stimulation of the sphenopalatine ganglion on cortical blood flow in the rat,” Journal of Cerebral Blood Flow and Metabolism, 8:875-878 (1988)
Van de Waterbeemd H, Camenisch G, Folkers G, Chretien J R, Raevsky O A, “Estimation of blood brain barrier crossing of drugs using molecular size and shape and h bonding descriptors,” Journal of Drug Targeting,” 6:151-165 (1998)
Suzuki N, Hardebo JE, Kahrstrom J, Owman C, “Selective electrical stimulation of postganglionic cerebrovascular parasympathetic nerve fibers originating from the sphenopalatine ganglion enhances cortical blood flow in the rat,” Journal of Cerebral Blood Flow and Metabolism 10:383-391 (1990)
Suzuki N, Hardebo J E, Kahrstrom J, Owman C H, “Effect on cortical blood flow of electrical stimulation of trigeminal cerebrovascular nerve fibres in the rat,” Acta Physiol Scand 138:307-315 (1990)
Fusco B M, Fiore G, Gallo F, Martelletti P, Giacovazzo M, “‘Capsaicin-sensitive’ sensory neurons in cluster headache: pathophysiological aspects and therapeutic indications,” Headache 34:132-137 (1994)
Lambert G A, Bogduk N, Goadsby P J, Duckworth J W, Lance J W, “Decreased carotid arterial resistance in cats in response to trigeminal stimulation,” Journal of Neurosurgery 61:307-315 (1984)

Nitric oxide (NO) is a gaseous free radical that is critical to numerous biological processes, including vasodilation, neurotransmission, immune stimulation, and smooth muscle relaxation. NO is generated endogenously almost exclusively by the oxidation of L-arginine, as catalyzed by NO synthases (NOS) (see, for example, Weisinger H, “Arginine metabolism and the synthesis of NO in the nervous system,” Progress in Neurobiology 64:365-391 (2001), which is incorporated herein by reference). NO has been used as an active ingredient in numerous drugs. Because NO is highly reactive and unstable, drugs typically comprise NO precursors or donors rather than NO itself. Wang P G et al., in “NO donors: chemical activities and biological applications,” Chem. Rev. 102:1091-1134 (2002), which is incorporated herein by reference, provide a list of some NO donors and some of their specific applications.

NO facilitators have been used as drugs for effecting vasodilation, including cerebral vasodilation. See, for example, the following articles, which are incorporated herein by reference:
Toda N et al., “Cerebral vasodilatation induced by stimulation of the pterygopalatine ganglion and greater petrosal nerve in anesthetized monkeys,” Neuroscience 96(2):393-398 (2000)
Toda N et al., “The pharmacology of NO in the peripheral nervous system of blood vessels,” Pharmacol Rev 55:271-324 (2003)
Toda N et al., “Neurogenic nitric oxide (NO) in the regulation of cerebroarterial tone,” J Chem Neuroanat 10(3-4):259-65 (1996).

US Patent Application Publication 2002/0155173 to Chopp et al., which is incorporated herein by reference, describes a method for promoting neurogenesis by administering a therapeutic amount of an NO donor compound to a patient. Also described are a compound for providing neurogenesis having an effective amount of an NO donor sufficient to promote neurogenesis, and an NO compound for promoting neurogenesis. Two compounds, DETANONOate and SNAP, are described as appropriate and as “likely cross[ing] the blood brain barrier.”

U.S. Pat. No. 6,433,019 to Nawa, which is incorporated herein by reference, describes a neurotrophic factor secretagogue, in particular, a brain-derived neurotrophic factor (BDNF) secretagogue, which comprises as an active ingredient an NO donor. This compound is described as promoting the secretion of neurotrophic factors from mammalian central neural cells, and as possibly applicable to the treatment of diseases caused by neurotrophic factors, for example, neurodegenerative diseases.

Several researchers have demonstrated the effect of NO on BBB permeability.

PCT Publication WO 00/23102 to Reiss et al., which is incorporated herein by reference, describes a method for regulating the permeability of the BBB by administering an NOS-3 inhibitor to reduce the increased permeability of the BBB caused by a pathological condition, or by administering an NOS-3 activator or NO donor to increase the permeability of the BBB. By increasing the permeability of the BBB, a therapeutic or diagnostic compound can be delivered across this barrier into the CNS.

The following articles, which are incorporated herein by reference, may be of interest:
Shyamaladevi N et al., “Evidence that NO production increases gamma amino butyric acid permeability of blood-brain barrier,” Brain Research Bulletin, 57(2):231-236 (2002)
Mayhan W G, “NO donor-induced increase in permeability of the blood-brain barrier,” Brain Research 866:101-108 (2000)
Mayhan W G, “Role of NO in histamine-induced increases in permeability of the blood-brain barrier,” Brain Research 743:70-76 (1996)
Nakano S et al., “Increased brain tumor microvessel permeability after intracarotid bradykinin infusion is mediated by NO,” Cancer Res 56(17):4027-31 (1996)
Mayhan W G, “VEGF increases permeability of the blood-brain barrier via a NO synthase/cGMP-dependent pathway,” Am J Physiol 276:C1148-C1153 (1999).

Controlled and/or targeted drug delivery techniques often enhance drug safety and/or efficacy of a drug by controlling the rate and/or location of release of the drug. The active ingredient of the drug is encapsulated in a carrier system, such as a nano- or micro-particle, a cell, a cell ghost, a lipoprotein, a liposome, a micelle, a microbubble, a microsphere, or a microparticle made of insoluble or biodegradable natural or synthetic polymers. The drug is gradually released as the carrier degrades in the body. The rate of degradation of some carriers varies responsively to conditions in the body, such as temperature, pH level, and enzymatic activity. Techniques have been developed to externally regulate the rate of release of the drug, such as by applying energy to the carrier from a source external or internal to the body. For example, such energy could be in the form of radiation, a magnetic field, or ultrasonic energy. Such regulation has been used to release the drug at a specific location of interest, and to precisely control the timing of drug release.

U.S. Pat. No. 6,117,454 to Kreuter et al., which is incorporated herein by reference, describes a method for delivering drugs and diagnostic agents across the BBB or BNB, comprising incorporating the drugs or diagnostic agents into nanoparticles which have been fabricated in conventional ways. These nanoparticles are then coated with additional surfactant and given to the body of animals or humans.

US Patent Application Publication 2002/0034474 to Sabel et al., which is incorporated herein by reference, describes a composition and method of fabrication with which nanoparticles may be used as a tool to deliver drugs to a specific target within or on a mammalian body, including across the BBB. Specifically, by using stabilizers other than Dextran 70.000 during the polymerization process, surfactants, which are described as being deemed necessary coating material in the prior art, are no longer required. Many substances are described as being useful as stabilizers, including Dextran 12.000 and polysorbate 85. A drug is either incorporated into or adsorbed onto the stabilized nanoparticles. This drug/nanoparticle complex is then administered to the organism by any route, such as by oral application, injection or inhalation, whereupon the drug exerts its effect at the desired site of pharmacological action.

U.S. Pat. No. 5,846,565 to Brem et al., which is incorporated herein by reference, describes devices for localized delivery of a chemotherapeutic agent to solid tumors, which agent normally does not cross the BBB and is characterized by poor bioavailability and/or a short half-life in vivo. The devices consist of reservoirs which release drug over an extended period while at the same time preserving the bioactivity and bioavailability of the agent. The devices may consist of biodegradable polymeric matrixes, although reservoirs can also be formulated from non-biodegradable polymers or reservoirs connected to implanted infusion pumps. The devices are implanted within or immediately adjacent the tumors to be treated or the site where they have been surgically removed.

U.S. Pat. No. 6,372,250 to Pardridge, which is incorporated herein by reference, describes the conjugation of liposomes containing therapeutic genes to multiple BBB- and brain-cell-membrane-targeting agents, so as to provide transport of the encapsulated gene across the BBB and brain cell membrane. Once across the BBB and brain cell membrane, the encapsulated gene expresses the encoded therapeutic agent within the brain to provide treatment and diagnosis of disease.

US Patent Application Publication 2003/0077243 to Fitzhugh et al., which is incorporated herein by reference, describes NO-releasing polymers, which are characterized as extremely hydrophobic. The NO-releasing polymers provided are extensively cross-linked polyamine-derivatized divinylbenzene diazeniumdiolates. These polymers are described as being able to be loaded with extremely high NO levels and designed to release NO in manners that mimic natural biological systems. The NO-releasing extremely hydrophobic polymers provided are described as being able to maintain a sustained NO release for periods exceeding nine months.

U.S. Pat. No. 5,405,919 to Keefer et al., which is incorporated herein by reference, describes a polymeric composition capable of releasing NO, including a polymer and an NO-releasing N₂O₂— functional group bound to the polymer; pharmaceutical compositions including the polymeric composition; and methods for treating biological disorders in which dosage with NO is beneficial.

US Patent Application Publication 2002/0068365 to Kuhrts, which is incorporated herein by reference, describes various controlled-release pharmaceutical compositions that include an agent that enhances or modulates the endogenous production of NO in a mammal. Controlled-release pharmaceutical compositions of L-arginine, its salts, peptides, and biological equivalents, together with methods of using the compositions are described. Also described are controlled-release pharmaceutical compositions of botanical extracts that modulate or enhance the production of NO, either alone or in combination with L-arginine or its biological equivalent.

U.S. Pat. No. 5,994,444 to Trescony et al., which is incorporated herein by reference, describes a polymeric material formed from a biodegradable polymer matrix is impregnated with an NO donor for continuous release of NO upon hydration.

US Patent Application Publication 2002/0049183 to Yedgar et al., which is incorporated herein by reference, describes methods for treating disease based upon the medicinal use of lipids and phospholipids covalently bound to physiologically acceptable monomers or polymers. Phosphatidylethanolamine moieties conjugated to physiologically acceptable monomers and polymers (PE conjugates) are described as manifesting a range of pharmacological effects, including stabilizing cell membranes; limiting oxidative damage to cell and blood components; limiting cell proliferation, cell extravasation and (tumor) cell migratory behavior; suppressing immune responses; and attenuating physiological reactions to stress, as expressed in elevated chemokine levels.

U.S. Pat. No. 6,258,780 to Soreq et al., which is incorporated herein by reference, describes a pharmaceutical composition for facilitating passage of compounds through the BBB, comprising the agent ACHE-I4 readthrough splice variant or the I4 peptide, and a pharmaceutically acceptable carrier. Alternatively, the pharmaceutical composition for facilitating passage of compounds through the BBB comprises the agents adrenaline, atropine, dopamine and/or an adrenergic combination and a pharmaceutically acceptable carrier. The composition optionally includes the compound to be transported across the BBB. Alternatively, the compound is co-administered (simultaneously) with the composition or is administered at some point during the biologically effective period of the action of the composition.

U.S. Pat. No. 6,443,898 to Unger et al., which is incorporated herein by reference, describes therapeutic delivery systems comprising gaseous precursor-filled microspheres comprising a therapeutic agent. A method is described for the controlled delivery of therapeutic compounds to a region of a patient comprising: (i) administering to the patient temperature activated gaseous precursor-filled microspheres comprising a therapeutic compound; (ii) monitoring the microspheres using energy to determine the liquid to gas phase transition and the presence of the microspheres in the region; and (iii) rupturing the microspheres using energy to release the therapeutic compound in the region. The energy may include ultrasound, microwave energy, radiofrequency energy, magnetic induction oscillating energy, and light energy.

U.S. Pat. No. 5,580,575 to Unger et al., which is incorporated herein by reference, describes therapeutic drug delivery systems comprising gas-filled microspheres comprising a therapeutic agent. A method is described for controlled delivery of therapeutic compounds to a region of a patient, comprising: (i) administering to the patient gas-filled microspheres comprising a therapeutic compound; (ii) monitoring the microspheres using ultrasound to determine the presence of the microspheres in the region; and (iii) rupturing the microspheres using ultrasound to release the therapeutic compound in the region.

US Patent Application Publication 2003/0092667 to Tachibana et al., which is incorporated herein by reference, describes methods for delivering therapeutic compositions to a target site using a catheter which includes at least one ultrasound transducer coupled to an energy source. The therapeutic compositions include genetic material and the target site may be a DNA with affinity for the genetic material.

PCT Publication WO 03/034975 to Conston et al., which is incorporated herein by reference, describes a method for site-specific delivery of therapeutic or diagnostic agents to a region in a fluid-filled cavity, vessel or tissue using an agent-loaded microbubble population. The population has controlled fragility characterized by a uniform wall thickness to diameter ratio which defines the discrete threshold intensity value of ultrasonic power where microbubble rupture occurs in the population. The location of the microbubble population may be monitored by ultrasound to determine its presence at the region prior to application of the ultrasonic power to rupture to microbubbles. Suitable drugs are described as including endothelium acting agents such as NO and NO donors.

Gabikian P et al., in “Prevention of experimental cerebral vasospasm by intracranial delivery of an NO donor from a controlled-release polymer: toxicity and efficacy studies in rabbits and rats,” Stroke 33:2681-2686 (2002), which is incorporated herein by reference, investigated the toxicity and efficacy of a locally-delivered NO donor from a controlled-release polymer in preventing experimental cerebral vasospasm in rats and rabbits. Diethylenetriamine/NO (DETA/NO) was incorporated into controlled-release ethylene-vinyl acetate (EVAc) polymers, and the polymers were implanted directly in the brain, in order to bypass the BBB. The researchers found that treatment with DETA/NO-EVAc polymer resulted in a significant decrease in basilar artery vasospasm compared with no treatment or compared with treatment with blank EVAC polymer.

The following articles, which are incorporated herein by reference, may be of interest:
Miyazaki S et al., “External control of drug release: controlled release of insulin from a hydrophilic polymer implant by ultrasound irradiation in diabetic rats,” J Pharm Pharmacol 40(10):716-7 (1988)
Lang D R, et al., “A controlled NO-releasing compound: synthesis, molecular structure, spectroscopy, electrochemistry, and chemical reactivity of R,R,S,S-trans-[RuCl(NO)(cyclam)]2+(1,4,8,11-tetraazacyclotetradecane),” Inorg Chem 39(11):2294-300 (2000)
Shishido S M et al., “Thermal and photochemical NO release from S-nitrosothiols incorporated in Pluronic F127 gel: potential uses for local and controlled NO release,” Biomaterials 24(20):3543-3553 (2003)
Sershen S et al., “Implantable, polymeric systems for modulated drug delivery,” Advanced Drug Delivery Reviews 54:1225-1235 (2002)
Lavon I et al., “Mass transport enhancement by ultrasound in non-degradable polymeric controlled release systems,” Journal of Controlled Release 54:1-7 (1998)
Torchilin V P, “Structure and design of polymeric surfactant-based drug delivery systems,” Journal of Controlled Release 73:137-172 (2001)
Rapoport N, “Stabilization and activation of pluronic micelles for tumor-targeted drug delivery,” colloids and surfaces B: Biointerfaces 16:93-111 (1999)
Marin A et al., “Drug delivery in pluronic micelles: effect of high-frequency ultrasound on drug release from micelles and intracellular uptake,” Journal of Controlled Release 84:39-47 (2002)
Marin A et al., “Acoustic activation of drug delivery from polymeric micelles: effect of pulsed ultrasound,” Journal of Controlled Release 71:239-249 (2001)
Nelson J L et al., “Ultrasonically Activated Chemotherapeutic Drug Delivery in a Rat Model,” Cancer Research 62:7280-7283 (2002)
Chiroli V et al., “NO-donating non-steroidal anti-inflammatory drugs: the case of nitroderivatives of aspirin,” European Journal of Medicinal Chemistry 38:441-446 (2003).

### SUMMARY OF THE INVENTION

In some embodiments of the present invention, a method for delivering molecules to a central nervous system (CNS) of a subject comprises supplying, to a body of the subject, (a) the molecules and (b) a nitric oxide (NO) facilitator encapsulated in a carrier system. Energy is applied to the carrier system so as to cause the carrier system to release the NO facilitator in a vicinity of a blood-brain barrier (BBB) of the subject. Such controlled and targeted release of the NO facilitator in the vicinity of the BBB increases permeability of the BBB and passage of the molecules from the blood circulation of the brain, through the BBB, and into the CNS of the subject. “NO facilitator,” as used in the present patent application and in the claims, consists of NO, NO precursors, NO donors, and any other molecule that facilitates the production of NO.

The molecules typically comprise a pharmaceutical agent or a diagnostic agent. For some applications, the molecules are encapsulated together with the NO facilitator in the carrier system. Alternatively, the molecules are encapsulated in another carrier system, or delivered using conventional delivery techniques, such as per-oral administration, intravenous administration, intra-arterial administration, intraperitoneal administration, subcutaneous administration, or intramuscular administration. Delivery of such molecules to the CNS can be beneficial for treating conditions such as ischemic conditions, vasospasm of CNS blood vessels, infections, primary CNS tumors, metastases in the CNS, Alzheimer's disease, Parkinson's disease, epilepsy, multiple sclerosis, Lou Gehrig's Disease (ALS), corticobasal degeneration (CBD), other neurodegenerative disorders, other conditions of the CNS, conditions of the eye, and conditions of the ear.

Targeting the release of the NO facilitator in the vicinity of the BBB typically avoids any undesirable effects of systemic release of NO. Such targeting also typically ensures that released NO, which is unstable, rapidly reaches the BBB while still active, and in concentrations sufficient to increase permeability of the BBB.

In some embodiments of the present invention, an NO facilitator encapsulated in a carrier system is supplied to the body of the subject, and energy is applied to the carrier system so as to cause the carrier system to release the NO facilitator in a vicinity of the BBB, and thereby cause a controlled increase in BBB permeability, vasodilation of CNS blood vessels and/or an increase in CNS blood flow. (“CNS blood flow” includes both cephalic blood flow and spinal cord blood flow. “Cephalic blood flow” includes cerebral and cerebellar blood flow.) Such increased vasodilation, permeability and/or CNS blood flow are generally beneficial for treating cerebrovascular disorders such as acute ischemic stroke and cerebral vasospasms after subarachnoid hemorrhage. For example, such treatment may increase survival of neuronal tissue during and following an ischemic event. Such increased vasodilation, permeability, and/or CNS blood flow are also generally beneficial for treating CNS disorders, such as vasospasm of CNS blood vessels, Alzheimer's disease, Parkinson's disease, epilepsy, multiple sclerosis, schizophrenia, depression, stress, obesity, pain, anxiety, and any other CNS disorder that is directly or indirectly affected by changes in CNS blood flow, vasodilation, or BBB permeability changes. For example, increased CNS blood flow may improve brain tissue metabolic state.

In some embodiments of the present invention, an NO facilitator encapsulated in a carrier system is supplied to the body of the subject, and energy is applied to the carrier system so as to cause the carrier system to release the NO facilitator in a vicinity of the BBB, and thereby increase clearance of a CNS constituent related to a CNS disorder, from the CNS, through the BBB, and into a systemic blood circulation of the subject. Such increased clearance is considered to be potentially beneficial for treating CNS disorders by lowering the concentration of the CNS-disorder-related constituent in the CNS, which typically reduces the biochemical burden of the constituent. CNS disorders for which this treatment method can be beneficial include, but are not limited to, glaucoma, macular edema, Gaucher's disease, late-onset Tay-Sachs, diabetic retinopathy, vasospasm of CNS blood vessels, Huntington's disease, Alzheimer's disease, Parkinson's disease, epilepsy, multiple sclerosis, ALS, CBD, other neurodegenerative disorders, and other conditions of the CNS.

In some embodiments of the present invention, a method for diagnosing a CNS disorder comprises supplying an NO facilitator encapsulated in a carrier system to the body of the subject, and applying energy to the carrier system so as to cause the carrier system to release the NO facilitator in a vicinity of the BBB, and thereby increase clearance of a CNS constituent related to the CNS disorder, from the CNS, through the BBB, and into a systemic blood circulation of the subject. Once in the systemic blood circulation, the CNS constituent is analyzed in order to facilitate a diagnosis of the CNS disorder. CNS disorders for which this diagnostic method can be useful include, but are not limited to, glaucoma, macular edema, Gaucher's disease, late-onset Tay-Sachs, diabetic retinopathy, Huntington's disease, Alzheimer's disease, Parkinson's disease, epilepsy, multiple sclerosis, ALS, CBD, and other neurodegenerative disorders.

In some embodiments of the present invention, a method for treating a disorder of a subject comprises supplying, to the body of the subject, an NO inhibitor or antagonist encapsulated in a carrier system, and applying energy to the carrier system so as to cause the carrier system to release the NO inhibitor/antagonist in a vicinity of a BBB of the subject, so as to decrease permeability of the BBB, thereby treating the disorder.

In some embodiments of the present invention, the carrier system comprising the NO facilitator is supplied to the body of the subject by supplying the carrier system to a systemic blood circulation of the subject, such as by per-oral administration, intravenous administration, intra-arterial administration, intraperitoneal administration, subcutaneous administration, or intramuscular administration. The carrier system circulates from the systemic blood circulation to a blood circulation of the CNS of the subject. In other embodiments, the carrier system is implanted in a vicinity of a blood vessel leading to the brain. Upon application of the energy, the carrier system releases the NO facilitator in the blood vessel, which is in the vicinity of the BBB.

In some embodiments of the present invention, the techniques described herein are applied to treat a disorder of or related to an eye of the subject. Such disorders include, but are not limited to, retinal vein occlusion and glaucoma. For treating such eye disorders, an NO facilitator or an NO inhibitor/antagonist is encapsulated in a carrier system and supplied to the body of the subject. Energy is applied to the carrier system, typically in a vicinity of the eye, so as to cause the carrier system to release the NO facilitator or NO inhibitor/antagonist in a vicinity of the eye, so as to increase or decrease permeability of the BBB in the vicinity of the eye, thereby treating the disorder.

In some embodiments of the present invention, the techniques described herein are applied to treat a disorder of or related to an ear of the subject.

There is therefore provided, in accordance with an embodiment of the present invention, a method for delivering molecules to a central nervous system (CNS) of a subject, the method including:
supplying the molecules to a blood circulation of the CNS;
supplying, to a body of the subject, a carrier system that encapsulates a nitric oxide (NO) facilitator; and
applying energy to the carrier system at an energy level sufficient to cause the carrier system to release the NO facilitator in a blood circulation of the subject in a vicinity of a blood-brain barrier (BBB) of the subject and thereby increase passage of the molecules from the blood circulation of the CNS, through the BBB, and into the CNS of the subject.

For some applications, supplying the molecules to the blood circulation of the CNS includes supplying the molecules to a blood circulation of a brain of the subject. For some applications, supplying the molecules to the blood circulation of the CNS includes supplying the molecules to a blood circulation of a spinal cord of the subject. For some applications, supplying the molecules includes administering the molecules to a systemic blood circulation of the subject.

In an embodiment, the energy is selected from the list consisting of: microwave energy, radiofrequency energy, and magnetic induction oscillating energy, and applying the energy includes applying the selected energy. In an embodiment, the energy includes light energy, and applying the energy includes applying the light energy.

For some applications, the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers, and supplying the carrier system includes supplying the selected carrier system. For other applications, the carrier system is selected from the list consisting of: a cell, a cell ghost, a lipoprotein, and a liposome, and supplying the carrier system includes supplying the selected carrier system.

In an embodiment, supplying the molecules includes selecting molecules effective in treating a condition selected from the list consisting of: an ischemic condition, vasospasm of a blood vessel of the CNS, infection, a CNS condition, a primary tumor of the CNS, and metastases in the CNS. In an embodiment, supplying the molecules includes selecting molecules effective in treating a condition selected from the list consisting of: pain and lower-back pain. In an embodiment, supplying the molecules includes selecting molecules effective in treating a condition selected from the list consisting of: Alzheimer's disease, Parkinson's disease, epilepsy, multiple sclerosis, Lou Gehrig's Disease (ALS), corticobasal degeneration (CBD), and a neurodegenerative disorder.

In an embodiment, applying the energy includes applying the energy from an energy applicator incorporated in a chair, in which the subject sits. Alternatively or additionally, applying the energy includes applying the energy from an energy applicator incorporated in a belt, which the subject wears.

In an embodiment, the energy includes ultrasound energy, and applying the energy includes applying the ultrasound energy.

For some applications, applying the energy includes applying the energy to a back of the subject.

In an embodiment, supplying the molecules includes selecting molecules effective in treating a condition of an eye of the subject. For some applications, the energy includes ultrasound energy, and applying the energy includes applying the ultrasound energy.

In an embodiment, supplying the molecules includes selecting molecules effective in treating a condition of an ear of the subject. For some applications, the energy includes ultrasound energy, and applying the energy includes applying the ultrasound energy.

In an embodiment, the molecules include a pharmaceutical agent, and supplying the molecules includes supplying the pharmaceutical agent. For some applications, the pharmaceutical agent includes an analgesic, and supplying the pharmaceutical agent includes supplying the analgesic. In an embodiment, the pharmaceutical agent is selected from the list consisting of: a neuroprotective agent, an enzyme, a chemotherapy agent, a virus that is a vector of gene therapy, an antiviral agent, an antibacterial agent, a glutamate receptor antagonist, an NMDA receptor blocker, a cholinesterase inhibitor, an agent having an inhibitory effect on derivation of β-amyloid from amyloid precursor protein, a β-amyloid inhibitor, an inhibitor of protein tyrosine phosphatases, a stimulant of nerve regeneration, a nerve growth factor, a compound that stimulates production of nerve growth factor, a microglial activation modulator, an antioxidant, a hormone, a medium chain triglyceride, an endogenous protein, a gene therapy agent, an anti-inflammatory agent, a non-steroidal anti-inflammatory drug (NSAID), a vaccine, a vaccine which includes antibodies against a specific protein that is characteristic of a disorder of the subject, a vaccine which includes antibodies against β-amyloid, a vaccine which includes antibodies against tau protein, a combination of a vaccine and an anti-inflammatory drug, a component of a vaccine, and a derivative of a vaccine, and supplying the pharmaceutical agent includes supplying the selected pharmaceutical agent.

In an embodiment, the molecules include a diagnostic agent, and supplying the molecules includes supplying the diagnostic agent. For some applications, the diagnostic agent includes an agent for facilitating diagnostic imaging, and supplying the diagnostic agent includes supplying the agent for facilitating diagnostic imaging. Alternatively, the diagnostic agent includes an antibody, and supplying the diagnostic agent includes supplying the antibody.

In an embodiment, the molecules are encapsulated in the carrier system, and supplying the molecules includes supplying the carrier system to the body. For some applications, the molecules are mixed with the NO facilitator, and supplying the molecules includes supplying the carrier system to the body. For some applications, the molecules are chemically conjugated with the NO facilitator, and supplying the molecules includes supplying the carrier system to the body.

In an embodiment, supplying the carrier system includes implanting the carrier system in a vicinity of a blood vessel of an upper circulation of the subject, which blood vessel supplies blood to a brain of the subject. For some applications, implanting the carrier system includes implanting the carrier system in an artery of the subject selected from the list consisting of: a carotid artery of the subject and a vertebral artery of the subject.

In an embodiment, supplying the carrier system includes administering the carrier system to a systemic blood circulation of the subject. For some applications, applying the energy includes applying the energy in a vicinity of an eye of the subject. For some applications, the energy includes light energy, and applying the energy includes applying the light energy. Alternatively or additionally, the energy includes ultrasound energy, and applying the energy includes applying the ultrasound energy. For some applications, applying the energy includes exposing the subject to ambient light.

For some applications, applying the energy includes applying the energy to substantially an entire brain of the subject. Alternatively, applying the energy includes targeting the energy to a specific area of a brain of the subject. For some applications, targeting the energy includes targeting the energy to an area of the BBB in a vicinity of a tumor.

There is also provided, in accordance with an embodiment of the present invention, a method for treating a central nervous system (CNS) disorder of a subject, the method including:
supplying, to a body of the subject, a carrier system that encapsulates a nitric oxide (NO) facilitator; and
applying energy to the carrier system at an energy level sufficient to cause the carrier system to release the NO facilitator in a blood circulation of the subject in a vicinity of a CNS of the subject and thereby cause vasodilation of CNS brain blood vessels and an increase in CNS blood flow, so as to treat the CNS disorder.

In an embodiment, applying the energy includes configuring the application of the energy to cause the carrier system to release the NO facilitator in the blood circulation in a vicinity of a brain of the subject. In an embodiment, applying the energy includes configuring the application of the energy to cause the carrier system to release the NO facilitator in the blood circulation in a vicinity of a spinal cord of the subject.

In an embodiment, the CNS disorder includes a disorder of an eye of the subject, and applying the energy includes applying the energy so as to treat the eye disorder.

In an embodiment, the CNS disorder includes a disorder of an ear of the subject, and applying the energy includes applying the energy so as to treat the ear disorder.

In an embodiment, the energy is selected from the list consisting of: microwave energy, radiofrequency energy, and magnetic induction oscillating energy, and applying the energy includes applying the selected energy. In an embodiment, the energy includes light energy, and applying the energy includes applying the light energy. In an embodiment, the energy includes ultrasound energy, and applying the energy includes applying the ultrasound energy.

In an embodiment, the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers, and supplying the carrier system includes supplying the selected carrier system. In an embodiment, the carrier system is selected from the list consisting of: a cell, a cell ghost, a lipoprotein, and a liposome, and supplying the carrier system includes supplying the selected carrier system.

In an embodiment, the CNS disorder includes a disorder of the brain of the subject, and applying the energy includes applying the energy so as to treat the brain disorder. In an embodiment, the CNS disorder is selected from the list consisting of: vasospasm of a blood vessel of the CNS, Gaucher's disease, late-onset Tay-Sachs, Huntington's disease, Alzheimer's disease, Parkinson's disease, epilepsy, multiple sclerosis, and schizophrenia, and applying the energy includes applying the energy so as to treat the selected CNS disorder. In an embodiment, the CNS disorder is selected from the list consisting of: glaucoma, macular edema, and diabetic retinopathy, and applying the energy includes applying the energy so as to treat the selected CNS disorder. In an embodiment, the CNS disorder is selected from the list consisting of: depression, stress, obesity, pain, and anxiety, and applying the energy includes applying the energy so as to treat the selected CNS disorder.

In an embodiment, supplying the carrier system includes administering the carrier system to a systemic blood circulation of the subject.

In an embodiment, the CNS disorder includes a vascular disorder of the CNS, and applying the energy includes applying the energy so as to treat the CNS vascular disorder. For some applications, the CNS vascular disorder includes cerebral vasospasms after subarachnoid hemorrhage of the subject, and applying the energy includes applying the energy so as to treat the cerebral vasospasms.

In an embodiment, supplying the carrier system includes implanting the carrier system in a vicinity of a blood vessel of an upper circulation of the subject, which blood vessel supplies blood to a brain of the subject. For some applications, implanting the carrier system includes implanting the carrier system in an artery of the subject selected from the list consisting of: a carotid artery of the subject and a vertebral artery of the subject.

In an embodiment, the CNS disorder includes an ischemic disorder of the subject, and applying the energy includes applying the energy at a level sufficient to cause vasodilation and thereby treat the ischemic disorder. For some applications, the ischemic disorder is selected from the list consisting of: arterial vein occlusion and vein thrombosis, and applying the energy includes applying the energy so as to treat the selected ischemic disorder. For some applications, the ischemic disorder includes retinal vein occlusion, and applying the energy includes applying the energy so as to treat the retinal vein occlusion. For some applications, the ischemic disorder includes a chronic ischemic disorder of the subject, and applying the energy includes applying the energy so as to treat the chronic ischemic disorder. For some applications, the ischemic disorder includes an acute ischemic event of the subject, and applying the energy includes applying the energy so as to treat the acute ischemic event. For some applications, the acute ischemic event includes acute ischemic stroke of the subject, and applying the energy includes applying the energy so as to treat the acute ischemic stroke.

There is further provided, in accordance with an embodiment of the present invention, a method for treating a disorder of a central nervous system (CNS) of a subject, the method including:
supplying, to a body of the subject, a carrier system encapsulating a nitric oxide (NO) facilitator; and
applying energy to the carrier system at an energy level sufficient to cause the carrier system to release the NO facilitator in a blood circulation of the subject in a vicinity of a blood-brain barrier (BBB) of the subject and thereby increase clearance of a CNS constituent related to the CNS disorder, from the CNS, through the BBB, and into a systemic blood circulation of the subject, so as to treat the CNS disorder.

In an embodiment, applying the energy includes configuring the energy to induce vasodilation, and thereby increase the clearance of the CNS constituent, to an extent that decreases edema of a brain of the subject.

In an embodiment, applying the energy includes configuring the energy to induce vasodilation, and thereby increase the clearance of the CNS constituent, to an extent that decreases edema of an eye of the subject.

In an embodiment, applying the energy includes configuring the energy to increase the clearance of the CNS constituent from a brain of the subject, through the BBB, and into the systemic blood circulation.

In an embodiment, applying the energy includes configuring the energy to increase the clearance of the CNS constituent from an eye of the subject, through the BBB, and into the systemic blood circulation.

In an embodiment, applying the energy includes configuring the energy to increase the clearance of the CNS constituent from a spinal cord of the subject, through the BBB, and into the systemic blood circulation.

In an embodiment, the CNS disorder is selected from the list consisting of: Gaucher's disease, late-onset Tay-Sachs, vasospasm of a blood vessel of the CNS, Huntington's disease, Alzheimer's disease, Parkinson's disease, epilepsy, multiple sclerosis, Lou Gehrig's Disease (ALS), and corticobasal degeneration (CBD), and applying the energy includes applying the energy so as to treat the selected CNS disorder. In an embodiment, the CNS disorder is selected from the list consisting of: glaucoma, macular edema, and diabetic retinopathy, and applying the energy includes applying the energy so as to treat the selected CNS disorder.

In an embodiment, the energy is selected from the list consisting of: microwave energy, radiofrequency energy, and magnetic induction oscillating energy, and applying the energy includes applying the selected energy. In an embodiment, the energy includes ultrasound energy, and applying the energy includes applying the ultrasound energy. In an embodiment, the energy includes light energy, and applying the energy includes applying the light energy.

In an embodiment, the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers, and supplying the carrier system includes supplying the selected carrier system. In an embodiment, the carrier system is selected from the list consisting of: a cell, a cell ghost, a lipoprotein, and a liposome, and supplying the carrier system includes supplying the selected carrier system.

In an embodiment, supplying the carrier system includes administering the carrier system to a systemic blood circulation of the subject.

In an embodiment, supplying the carrier system includes implanting the carrier system in a vicinity of a blood vessel of an upper circulation of the subject, which blood vessel supplies blood to a brain of the subject. For some applications, implanting the carrier system includes implanting the carrier system in an artery of the subject selected from the list consisting of: a carotid artery of the subject and a vertebral artery of the subject.

There is yet further provided, in accordance with an embodiment of the present invention, a method for facilitating a diagnosis of a disorder of a central nervous system (CNS) of a subject, the method including:
supplying, to a body of the subject, a carrier system encapsulating a nitric oxide (NO) facilitator; and
applying energy to the carrier system at an energy level sufficient to cause the carrier system to release the NO facilitator in a blood circulation of the subject in a vicinity of a blood-brain barrier (BBB) of the subject and thereby increase clearance of a CNS constituent related to the CNS disorder, from the CNS, through the BBB, and into another body compartment of the subject, so as to facilitate the diagnosis of the CNS disorder.

In an embodiment, applying the energy includes configuring the energy to increase the clearance of the CNS constituent from a brain of the subject, through the BBB, and into the other body compartment. In an embodiment, applying the energy includes configuring the energy to increase the clearance of the CNS constituent from an eye of the subject, through the BBB, and into the other body compartment. In an embodiment, applying the energy includes configuring the energy to increase the clearance of the CNS constituent from a spinal cord of the subject, through the BBB, and into the other body compartment.

In an embodiment, the other body compartment includes a systemic blood circulation of the subject, and applying the energy includes setting the energy level to be sufficient to increase the clearance of the CNS constituent from the CNS to the systemic blood circulation. In an embodiment, the other body compartment includes plasma of the subject, and applying the energy includes setting the energy level to be sufficient to increase the clearance of the CNS constituent from the CNS to the plasma. In an embodiment, the other body compartment includes serum of the subject, and applying the energy includes setting the energy level to be sufficient to increase the clearance of the CNS constituent from the CNS to the serum. In an embodiment, the other body compartment is ascites of the subject, and applying the energy includes setting the energy level to be sufficient to increase the clearance of the CNS constituent from the CNS to the ascites.

In an embodiment, the CNS disorder is selected from the list consisting of: Gaucher's disease, late-onset Tay-Sachs, vasospasm of a blood vessel of the CNS, Huntington's disease, Alzheimer's disease, Parkinson's disease, a tumor, epilepsy, multiple sclerosis, Lou Gehrig's Disease (ALS), and corticobasal degeneration (CBD). In an embodiment, the CNS disorder is selected from the list consisting of: glaucoma, macular edema, and diabetic retinopathy.

In an embodiment, the energy is selected from the list consisting of: microwave energy, radiofrequency energy, and magnetic induction oscillating energy, and applying the energy includes applying the selected energy. In an embodiment, the energy includes ultrasound energy, and applying the energy includes applying the ultrasound energy. In an embodiment, the energy includes light energy, and applying the energy includes applying the light energy.

In an embodiment, the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers, and supplying the carrier system includes supplying the selected carrier system. In an embodiment, the carrier system is selected from the list consisting of: a cell, a cell ghost, a lipoprotein, and a liposome, and supplying the carrier system includes supplying the selected carrier system.

In an embodiment, supplying the carrier system includes administering the carrier system to a systemic blood circulation of the subject. In an embodiment, supplying the carrier system includes implanting the carrier system in a vicinity of a blood vessel of an upper circulation of the subject, which blood vessel supplies blood to a brain of the subject. For some applications, implanting the carrier system includes implanting the carrier system in an artery of the subject selected from the list consisting of: a carotid artery of the subject and a vertebral artery of the subject.

There is still further provided, in accordance with an embodiment of the present invention, a method for treating a disorder of a subject, the method including:
supplying, to a body of the subject, a carrier system encapsulating a nitric oxide (NO) inhibitor; and
applying energy to the carrier system at an energy level sufficient to cause the carrier system to release the NO inhibitor in a blood circulation of the subject in a vicinity of a blood-brain barrier (BBB) of the subject and thereby decrease permeability of the BBB, so as to treat the disorder.

In an embodiment, the disorder includes multiple sclerosis, and applying the energy includes applying the energy so as to treat the multiple sclerosis. In an embodiment, the disorder includes migraine headache, and applying the energy includes applying the energy so as to treat the migraine headache. In an embodiment, the disorder includes neuroinflammation, and applying the energy includes applying the energy so as to treat the neuroinflammation. In an embodiment, the disorder includes damage caused to the BBB by infection, and applying the energy includes applying the energy so as to treat the damage. In an embodiment, the disorder includes damage caused to the BBB by a bacterial toxin, and applying the energy includes applying the energy so as to treat the damage.

In an embodiment, the energy is selected from the list consisting of: microwave energy, radiofrequency energy, and magnetic induction oscillating energy, and applying the energy includes applying the selected energy. In an embodiment, the energy includes ultrasound energy, and applying the energy includes applying the ultrasound energy. In an embodiment, the energy includes light energy, and applying the energy includes applying the light energy.

In an embodiment, the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers, and supplying the carrier system includes supplying the selected carrier system. In an embodiment, the carrier system is selected from the list consisting of: a cell, a cell ghost, a lipoprotein, and a liposome, and supplying the carrier system includes supplying the selected carrier system.

In an embodiment, supplying the carrier system includes implanting the carrier system in a vicinity of a blood vessel of an upper circulation of the subject, which blood vessel supplies blood to a brain of the subject. For some applications, implanting the carrier system includes implanting the carrier system in an artery of the subject selected from the list consisting of: a carotid artery of the subject and a vertebral artery of the subject.

In an embodiment, supplying the carrier system includes administering the carrier system to a systemic blood circulation of the subject. In an embodiment, applying the energy includes applying the energy to a vicinity of an eye of the subject.

There is additionally provided, in accordance with an embodiment of the present invention, a method for treating a disorder of a subject, the method including:
supplying, to the blood circulation of a brain of the subject, a light-activated nitric oxide (NO) precursor; and
applying light to the NO precursor at a level sufficient to cause the NO precursor to release NO in a vicinity of a blood-brain barrier (BBB) of the subject and thereby increase permeability of the BBB, so as to treat the disorder.

In an embodiment, the method includes supplying molecules to the blood circulation of the brain, and applying the light includes configuring the light applied to the NO precursor to be of a level sufficient to cause the NO precursor to release the NO and thereby increase passage of the molecules from the blood circulation of the brain, through the BBB, and into the CNS of the subject.

In an embodiment, applying the light includes applying the light through an eye of the subject.

In an embodiment, the NO precursor is selected from the list consisting of: a nitrosothiol, an organic nitrite, an N-nitrosamine, and a nitrosamine, and supplying the NO precursor includes supplying the selected NO precursor.

In an embodiment, the disorder includes a disorder of an eye of the subject, and applying the light includes applying the light so as to treat the eye disorder.

In an embodiment, the disorder includes a disorder of an ear of the subject, and applying the light includes applying the light so as to treat the ear disorder.

In an embodiment, the method includes: supplying, to a body of the subject, a carrier system encapsulating the light-activated NO precursor; and applying energy to the carrier system at an energy level sufficient to cause the carrier system to release the light-activated NO facilitator in a blood circulation of the subject in a vicinity of the BBB. For some applications, applying the energy includes applying the light. For some applications, the energy is selected from the list consisting of: microwave energy, radiofrequency energy, and magnetic induction oscillating energy, and applying the energy includes applying the selected energy. For some applications, the energy includes ultrasound energy, and applying the energy includes applying the ultrasound energy. For some applications, supplying the carrier system includes implanting the carrier system in a vicinity of a blood vessel of an upper circulation of the subject, which blood vessel supplies blood to the brain. For some applications, implanting the carrier system includes implanting the carrier system in an artery of the subject selected from the list consisting of: a carotid artery of the subject and a vertebral artery of the subject.

In an embodiment, supplying the carrier system includes administering the carrier system to a systemic blood circulation of the subject. In an embodiment, applying the energy includes applying the energy in a vicinity of the eye.

There yet additionally provided, in accordance with an embodiment of the present invention, a method for treating a disorder of a subject, including:
supplying, to a body of the subject, a carrier system encapsulating a nitric oxide (NO) facilitator; and
applying energy to the carrier system at an energy level sufficient to cause the carrier system to release the NO facilitator in a blood circulation of the subject in a vicinity of a blood-brain barrier (BBB) of the subject and thereby increase passage of a substance through the BBB between a spinal cord of the subject and the blood circulation, so as to treat the disorder.

In an embodiment, the energy is selected from the list consisting of: microwave energy, radiofrequency energy, and magnetic induction oscillating energy, and applying the energy includes applying the selected energy.

In an embodiment, the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers, and supplying the carrier system includes supplying the selected carrier system. In an embodiment, the carrier system is selected from the list consisting of: a cell, a cell ghost, a lipoprotein, and a liposome, and supplying the carrier system includes supplying the selected carrier system.

In an embodiment, applying the energy includes applying the energy from an energy applicator incorporated in a chair, in which the subject sits. In an embodiment, applying the energy includes applying the energy from an energy applicator incorporated in a belt, which the subject wears.

In an embodiment, the substance includes a constituent of the spinal cord, and applying the energy includes configuring the energy to increase clearance of the constituent from the spinal cord, through the BBB, to the blood circulation.

In an embodiment, the energy includes ultrasound energy, and applying the energy includes applying the ultrasound energy. In an embodiment, the energy includes light energy, and applying the energy includes applying the light energy.

In an embodiment, applying the energy includes applying the energy to a back of the subject.

In an embodiment, the substance includes molecules, and the method includes supplying the molecules to the blood circulation, and applying the energy includes configuring the energy to increase passage of the molecules from the blood circulation, through the BBB, to the spinal cord.

In an embodiment, the molecules include a diagnostic agent, and supplying the molecules includes supplying the diagnostic agent. In an embodiment, the molecules include a pharmaceutical agent, and supplying the molecules includes supplying the pharmaceutical agent. For some applications, the pharmaceutical agent includes an analgesic, and supplying the pharmaceutical agent includes supplying the analgesic.

In an embodiment, the molecules are encapsulated in the carrier system, and supplying the molecules includes supplying the carrier system to the body. In an embodiment, the molecules are mixed with the NO facilitator, and supplying the molecules includes supplying the carrier system to the body. In an embodiment, the molecules are chemically conjugated with the NO facilitator, and supplying the molecules includes supplying the carrier system to the body.

There is still additionally provided, in accordance with an embodiment of the present invention, a method for facilitating a diagnosis of a disorder of a subject, including:
supplying, to a body of the subject, a carrier system encapsulating a nitric oxide (NO) facilitator; and
applying energy to the carrier system at an energy level sufficient to cause the carrier system to release the NO facilitator in a blood circulation of the subject in a vicinity of a blood-brain barrier (BBB) of the subject and thereby increase passage of a spinal cord constituent, from a spinal cord of the subject, through the BBB, and into a systemic blood circulation of the subject, so as to facilitate the diagnosis of the disorder.

There is also provided, in accordance with an embodiment of the present invention, a molecule delivery system including:
molecules adapted to be supplied to a blood circulation of a central nervous system (CNS) of a subject;
a carrier system adapted to be supplied to a body of the subject, the carrier system encapsulating a nitric oxide (NO) facilitator; and
a transducer, adapted to apply ultrasound energy to the carrier system at an energy level sufficient to cause the carrier system to release the NO facilitator in a blood circulation of the subject in a vicinity of a blood-brain barrier (BBB) of the subject and thereby increase passage of the molecules from the blood circulation of the CNS, through the BBB, and into the CNS of the subject.

In an embodiment, the transducer is adapted to apply the energy to a back of the subject.

In an embodiment, the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers. In an embodiment, the carrier system is selected from the list consisting of: a cell, a cell ghost, a lipoprotein, and a liposome.

In an embodiment, the molecules include molecules effective in treating a condition selected from the list consisting of: an ischemic condition, vasospasm of a blood vessel of the CNS, infection, a CNS condition, a primary tumor of the CNS, and metastases in the CNS. In an embodiment, the molecules include molecules effective in treating a condition selected from the list consisting of: pain and lower-back pain. In an embodiment, the molecules include molecules effective in treating a condition selected from the list consisting of: Alzheimer's disease, Parkinson's disease, epilepsy, multiple sclerosis, Lou Gehrig's Disease (ALS), corticobasal degeneration (CBD), and a neurodegenerative disorder.

In an embodiment, the molecules include molecules effective in treating a condition of an eye of the subject.

In an embodiment, the molecules include molecules effective in treating a condition of an ear of the subject.

In an embodiment, the molecule delivery system includes a chair, in which the transducer is incorporated, and which is adapted to be sat in by the subject.

In an embodiment, the molecule delivery system includes a belt, in which the transducer is incorporated, and which is adapted to be worn by the subject.

In an embodiment, the molecules include a pharmaceutical agent. In an embodiment, the pharmaceutical agent includes an analgesic. In an embodiment, the pharmaceutical agent is selected from the list consisting of: an analgesic agent, a neuroprotective agent, an enzyme, a chemotherapy agent, a virus that is a vector of gene therapy, an antiviral agent, an antibacterial agent, a glutamate receptor antagonist, an NMDA receptor blocker, a cholinesterase inhibitor, an agent having an inhibitory effect on derivation of β-amyloid from amyloid precursor protein, a β-amyloid inhibitor, an inhibitor of protein tyrosine phosphatases, a stimulant of nerve regeneration, a nerve growth factor, a compound that stimulates production of nerve growth factor, a microglial activation modulator, an antioxidant, a hormone, a medium chain triglyceride, an endogenous protein, a gene therapy agent, an anti-inflammatory agent, a non-steroidal anti-inflammatory drug (NSAID), a vaccine, a vaccine which includes antibodies against a specific protein that is characteristic of a disorder of the subject, a vaccine which includes antibodies against β-amyloid, a vaccine which includes antibodies against tau protein, a combination of a vaccine and an anti-inflammatory drug, a component of a vaccine, and a derivative of a vaccine.

In an embodiment, the molecules include a diagnostic agent. For some applications, the diagnostic agent includes an agent for facilitating diagnostic imaging. Alternatively, the diagnostic agent includes an antibody.

In an embodiment, the molecules are encapsulated in the carrier system. In an embodiment, the molecules are mixed with the NO facilitator. In an embodiment, the molecules are chemically conjugated with the NO facilitator.

In an embodiment, the carrier system is adapted to be implanted in a vicinity of a blood vessel of an upper circulation of the subject, which blood vessel supplies blood to a brain of the subject.

In an embodiment, the carrier system is adapted to be implanted in an artery of the subject selected from the list consisting of: a carotid artery of the subject and a vertebral artery of the subject.

In an embodiment, the carrier system is adapted to be administered to a systemic blood circulation of the subject.

In an embodiment, the transducer is adapted to apply the energy in a vicinity of an eye of the subject.

There is further provided, in accordance with an embodiment of the present invention, a treatment system for treating a central nervous system (CNS) disorder of a subject, the treatment system including:
a carrier system adapted to be supplied to a body of the subject, the carrier system encapsulating a nitric oxide (NO) facilitator; and
a transducer, adapted to apply ultrasound energy to the carrier system at an energy level sufficient to cause the carrier system to release the NO facilitator in a blood circulation of the subject in a vicinity of a blood-brain barrier (BBB) of the subject and thereby cause vasodilation of CNS blood vessels and an increase in CNS blood flow, so as to treat the CNS disorder.

In an embodiment, the CNS disorder includes a disorder of an eye of the subject, and the transducer is configured to apply the energy so as to treat the eye disorder.

In an embodiment, the CNS disorder includes a disorder of an ear of the subject, and the transducer is configured to apply the energy so as to treat the ear disorder.

In an embodiment, the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers. In an embodiment, the carrier system is selected from the list consisting of: a cell, a cell ghost, a lipoprotein, and a liposome.

In an embodiment, the CNS disorder includes a disorder of a brain of the subject, and the transducer is configured to apply the energy so as to treat the brain disorder. In an embodiment, the CNS disorder is selected from the list consisting of: vasospasm of a blood vessel of the CNS, Gaucher's disease, late-onset Tay-Sachs, Huntington's disease, Alzheimer's disease, Parkinson's disease, epilepsy, multiple sclerosis, and schizophrenia, and the transducer is configured to apply the energy so as to treat the selected CNS disorder. In an embodiment, the CNS disorder is selected from the list consisting of: glaucoma, macular edema, and diabetic retinopathy, and the transducer is configured to apply the energy so as to treat the selected CNS disorder. In an embodiment, the CNS disorder is selected from the list consisting of: depression, stress, obesity, pain, and anxiety, and the transducer is configured to apply the energy so as to treat the selected CNS disorder.

In an embodiment, the carrier system is adapted to be administered to a systemic blood circulation of the subject.

In an embodiment, the CNS disorder includes a vascular disorder of the CNS, and the transducer is configured to apply the energy so as to treat the CNS vascular disorder. In an embodiment, the CNS vascular disorder includes cerebral vasospasms after subarachnoid hemorrhage of the subject, and the transducer is configured to apply the energy so as to treat the cerebral vasospasms.

In an embodiment, the carrier system is adapted to be implanted in a vicinity of a blood vessel of an upper circulation of the subject, which blood vessel supplies blood to a brain of the subject. For some applications, the carrier system is adapted to be implanted in an artery of the subject selected from the list consisting of: a carotid artery of the subject and a vertebral artery of the subject.

In an embodiment, the CNS disorder includes an ischemic disorder of the subject, and the transducer is configured to apply the energy at a level sufficient to cause vasodilation and thereby treat the ischemic disorder. For some applications, the ischemic disorder is selected from the list consisting of: arterial vein occlusion and vein thrombosis, and the transducer is configured to apply the energy so as to treat the selected ischemic disorder. For some applications, the ischemic disorder includes retinal vein occlusion, and the transducer is configured to apply the energy so as to treat the retinal vein occlusion. For some applications, the ischemic disorder includes a chronic ischemic disorder of the subject, and the transducer is configured to apply the energy so as to treat the chronic ischemic disorder. For some applications, the ischemic disorder includes an acute ischemic event of the subject, and the transducer is configured to apply the energy so as to treat the acute ischemic event. For some applications, the acute ischemic event includes acute ischemic stroke of the subject, and the transducer is configured to apply the energy so as to treat the acute ischemic stroke.

There is yet further provided, in accordance with an embodiment of the present invention, a treatment system for treating a central nervous system (CNS) disorder of a subject, the treatment system including:
a carrier system adapted to be supplied to a body of the subject, the carrier system encapsulating a nitric oxide (NO) facilitator; and
a transducer, adapted to apply ultrasound energy to the carrier system at an energy level sufficient to cause the carrier system to release the NO facilitator in a blood circulation of the subject in a vicinity of a blood-brain barrier (BBB) of the subject and thereby increase clearance of a CNS constituent related to the CNS disorder, from the CNS, through the BBB, and into a systemic blood circulation of the subject, so as to treat the CNS disorder.

There is still further provided, in accordance with an embodiment of the present invention, a diagnostic system for facilitating a diagnosis of a disorder of a central nervous system (CNS) of a subject, the diagnostic system including:
a carrier system adapted to be supplied to a body of the subject, the carrier system encapsulating a nitric oxide (NO) facilitator; and
a transducer, adapted to apply ultrasound energy to the carrier system at an energy level sufficient to cause the carrier system to release the NO facilitator in a blood circulation of the subject in a vicinity of a blood-brain barrier (BBB) of the subject and thereby increase clearance of a CNS constituent related to the CNS disorder, from the CNS, through the BBB, and into another body compartment of the subject, so as to facilitate the diagnosis of the CNS disorder.

There is also provided, in accordance with an embodiment of the present invention, a treatment system for treating a disorder of a subject, the treatment system including:
a carrier system adapted to be supplied to a body of the subject, the carrier system encapsulating a nitric oxide (NO) inhibitor; and
a transducer, adapted to apply ultrasound energy to the carrier system at an energy level sufficient to cause the carrier system to release the NO inhibitor in a blood circulation of the subject in a vicinity of a blood-brain barrier (BBB) of the subject and thereby decrease permeability of the BBB, so as to treat the disorder.

There is additionally provided, in accordance with an embodiment of the present invention, a treatment system for treating a disorder of a subject, the treatment system including:
a light-activated nitric acid (NO) precursor; and
a light source, adapted to apply light to the NO precursor at a level sufficient to cause the NO precursor to release NO in a vicinity of a blood-brain barrier (BBB) of the subject and thereby increase permeability of the BBB, so as to treat the disorder.

There is still additionally provided, in accordance with an embodiment of the present invention, a treatment system for treating a disorder of a subject, the treatment system including:
a carrier system adapted to be supplied to a body of the subject, the carrier system encapsulating a nitric oxide (NO) facilitator; and
a transducer, adapted to apply ultrasound energy to the carrier system at an energy level sufficient to cause the carrier system to release the NO facilitator in a blood circulation of the subject in a vicinity of a blood-brain barrier (BBB) of the subject and thereby increase passage of a substance through the BBB between a spinal cord of the subject and the blood circulation, so as to treat the disorder.

There is also provided, in accordance with an embodiment of the present invention, a diagnostic system for facilitating a diagnosis of a disorder of a subject, the diagnostic system including:
a carrier system adapted to be supplied to a body of the subject, the carrier system encapsulating a nitric oxide (NO) facilitator; and
a transducer, adapted to apply ultrasound energy to the carrier system at an energy level sufficient to cause the carrier system to release the NO facilitator in a blood circulation of the subject in a vicinity of a blood-brain barrier (BBB) of the subject and thereby increase passage of a spinal cord constituent, from a spinal cord of the subject, through the BBB, and into a systemic blood circulation of the subject, so as to facilitate the diagnosis of the disorder.

The present invention will be more fully understood from the following detailed description of the embodiments thereof, taken together with the drawings in which:

### BRIEF DESCRIPTION OF THE DRAWINGS

FIG. 1 is a schematic illustration of an external energy applicator applied in a vicinity of a brain of a subject, in accordance with an embodiment of the present invention; and

FIG. 2 is a schematic illustration of a carrier system implanted in a vicinity of a blood vessel supplying blood to a brain of a subject, in accordance with an embodiment of the present invention.

### DETAILED DESCRIPTION OF EMBODIMENTS

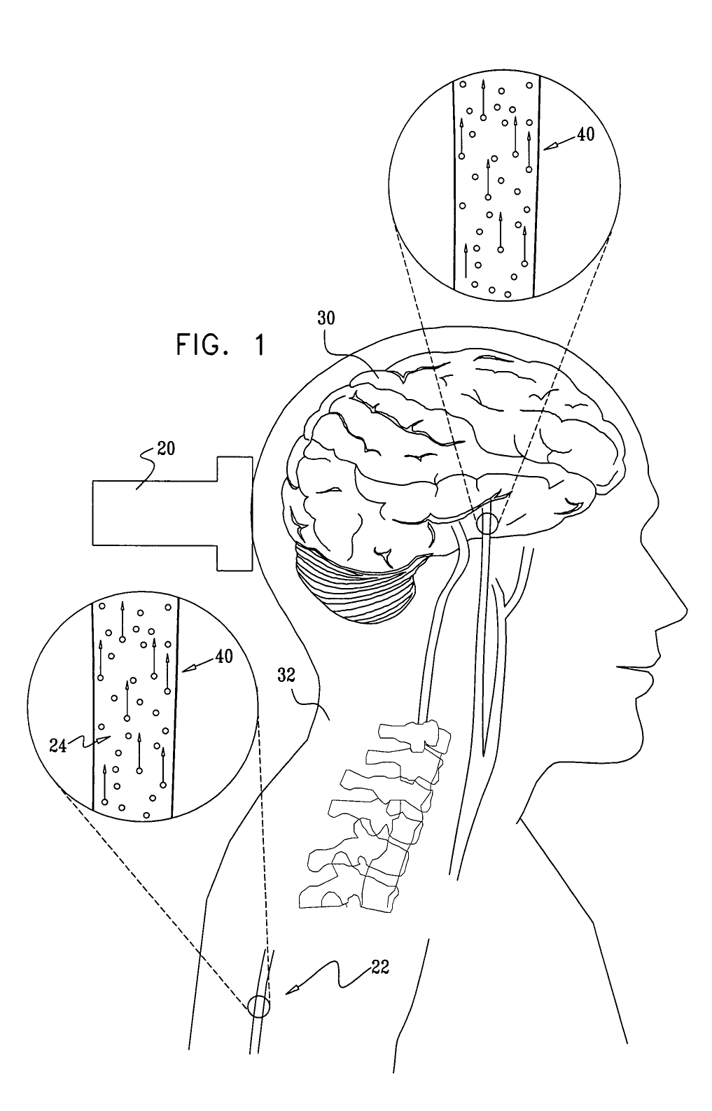

FIG. 1 is a schematic illustration of an energy applicator 20 applied in a vicinity of a portion of a central nervous system (CNS) of a subject 32, such as a brain 30 of subject 32, in accordance with an embodiment of the present invention. Energy applicator 20 is adapted to apply a controlled amount of energy to a carrier system 40, which encapsulates a nitric oxide (NO) facilitator. For example, such energy may comprise ultrasound energy, microwave energy, radiofrequency energy, magnetic induction oscillating energy, or light energy. Application of the energy causes carrier system 40 to release the NO facilitator in a blood circulation of subject 32 in a vicinity of a blood-brain barrier (BBB) of the subject, thereby increasing permeability of the BBB. Details of the design and manufacture of energy applicator 20 will be apparent to those skilled in the art, having read the present patent application.

In an embodiment of the present invention, carrier system 40 comprises a blood-borne carrier system 24, which is supplied to a systemic blood circulation 22 of subject 32. Such supplying may be performed, for example, by per-oral administration, intravenous administration, intra-arterial administration, intraperitoneal administration, subcutaneous administration, or intramuscular administration. Blood-borne carrier system 24 circulates from systemic blood circulation 22 to a blood circulation of brain 30. In this embodiment, energy applicator 20 is typically configured and/or positioned to target the energy to a specific area of brain 30 or substantially the entire brain, depending on the application.

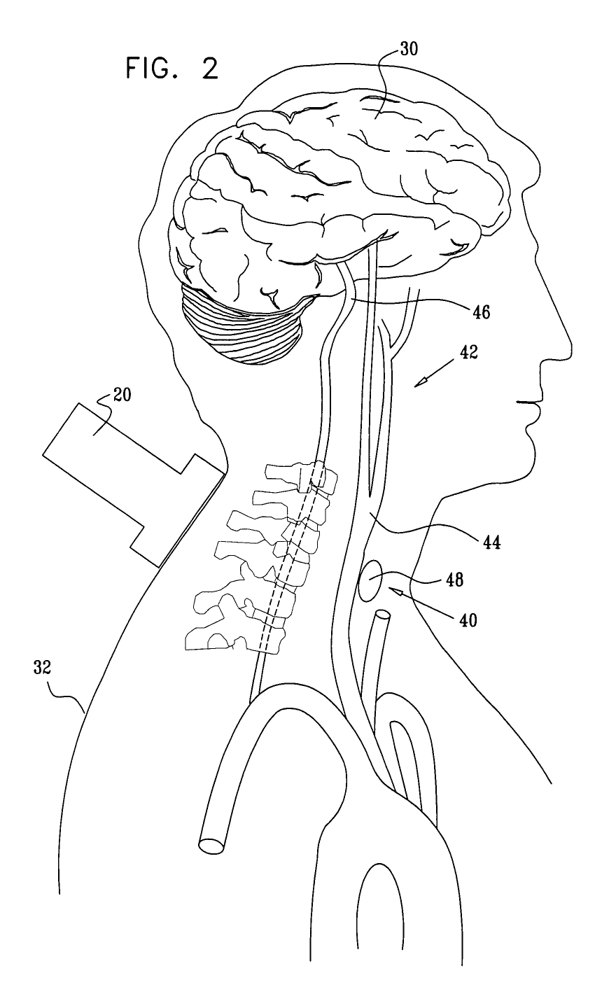

FIG. 2 is a schematic illustration of carrier system 40 implanted in a vicinity of a blood vessel 42 of an upper circulation of subject 32, which blood vessel supplies blood to brain 30, in accordance with an embodiment of the present invention. For example, blood vessel 42 may comprise a carotid artery 44 or a vertebral artery 46 of subject 32. In this embodiment, carrier system 40 comprises an implantable carrier system 48, which comprises a nitric oxide (NO) facilitator, and energy applicator 20 is typically configured and/or positioned to apply the energy in a vicinity of blood vessel 42. Upon application of the energy, the carrier system releases the NO facilitator in blood vessel 42, which is in a vicinity of the BBB.

“NO facilitator,” as used in the present patent application and in the claims, consists of NO, NO precursors, NO donors, and any other molecule that facilitates the production of NO. Numerous NO precursors and donors are known in the art, including those described in the patents, patent application publications, and articles cited hereinabove. For example, NO donors can be classified as described in the above-mentioned article by Wang et al.: organic nitrates, organic nitrites, metal-NO complexes, N-Nitrosamines, N-Hydroxyl-N-nitrosamines, nitrosimines, nitrosothiols (RSNOs), C-nitroso compounds, diazetine dioxides, furoxans and benzofuroxans, oxatriazole-5-imines, sydnonimines, oximes, hydroxylamines, N-hydroxyguanidines, and hydroxyureas. NO donors also include diazeniumdiolates, for example as described in the above-mentioned US Patent Application Publication 2003/0077243.

Carrier system 40 comprises a material which is adapted to encapsulate the NO facilitator and release the NO facilitator in substantial quantities only upon exposure to one or more specific types of energy, applied at a sufficient magnitude. For example, carrier system 40 may comprise a polymer or a nano- or micro-particle, a cell, a cell ghost, a lipoprotein, a liposome, a micelle, a microbubble, a microsphere, or a microparticle made of insoluble or biodegradable natural or synthetic polymers. Particles and/or methods for fabricating and/or using particles may be used that are described in the patents, patent application publications, and articles described hereinabove. Alternatively, other particles and/or methods for fabricating and/or using particles may be used that are known in the art.

By way of example and not limitation:
for applications in which carrier system 40 comprises implantable carrier system 48, the carrier system may comprise an ultrasound-sensitive bio-polymer in which the NO facilitator is encapsulated, as described, for example, in the above-referenced articles by Sershen S et al., Lavon I et al., Miyazaki S et al., Gabikian P et al., and/or Torchilin VP; and
for applications in which carrier system 40 comprises blood-borne carrier system 24, which is delivered to the blood circulation of brain 30 via systemic blood circulation 22, the carrier system may comprise (a) an ultrasound-sensitive stabilized pluronic micelle in which the NO facilitator is encapsulated, as described, for example, in the above-referenced articles by (i) Rapoport N, (ii) Marin A et al., and/or (iii) Nelson J L et al., or (b) a radiation-sensitive (e.g., electromagnetic radiation-sensitive) nano- or micro-particle, such as a liposome, in which the NO facilitator is encapsulated.

In an embodiment of the present invention, a method for delivery of a molecule to the CNS of subject 32 comprises supplying, to the body of subject 32, an NO facilitator encapsulated in carrier system 40 and the molecule. Energy applicator 20 is activated to apply energy to carrier system 40 so as to cause the carrier system to release the NO facilitator. The NO facilitator releases NO in a vicinity of the BBB, so as to increase passage of the molecules from the blood circulation of brain 30, through the BBB, and into the CNS of the subject. Delivery of such molecules to the CNS can be beneficial for treating conditions such as ischemic conditions, vasospasm of CNS blood vessels, infections, primary CNS tumors, metastases in the CNS, Alzheimer's disease, Parkinson's disease, epilepsy, multiple sclerosis, Lou Gehrig's Disease (ALS), corticobasal degeneration (CBD), other neurodegenerative disorders, other conditions of the CNS, conditions of the eye, and conditions of the ear.

For some applications, the molecules are encapsulated together with the NO facilitator in the same carrier system 40. For example, the molecules may be mixed with the NO facilitator. Alternatively, the molecules are chemically conjugated with the NO facilitator, which may have the added benefit of enhancing the efficacy of the molecules. For example, there are a number of NO-conjugated drugs, such as NO-NSAIDs and NO-aspirins, which have been shown to have enhanced efficacy. Alternatively, the molecules are encapsulated in another carrier system, or delivered using conventional delivery techniques, such as per-oral administration, intravenous administration, intra-arterial administration, intraperitoneal administration, subcutaneous administration, or intramuscular administration. In the case of conventional delivery techniques, energy applicator 20 is typically activated to release the NO facilitator when the molecules achieve a desired concentration in the blood circulation of brain 30, typically immediately or soon after administration of the molecules.

The molecules typically comprise a pharmaceutical agent or a diagnostic agent. Pharmaceutical agents appropriate for delivery using the techniques described herein include any pharmaceutical agent targeted at the CNS, including, but not limited to, an analgesic agent, a neuroprotective agent, an enzyme, a chemotherapy agent, a virus that is a vector of gene therapy (e.g., for treating Parkinson's disease), a glutamate receptor antagonist, an NMDA receptor blocker, a cholinesterase inhibitor, an agent having an inhibitory effect on derivation of β-amyloid from amyloid precursor protein, a β-amyloid inhibitor, an inhibitor of protein tyrosine phosphatases, a stimulant of nerve regeneration, a nerve growth factor, a compound that stimulates production of nerve growth factor, a microglial activation modulator, an antioxidant, a hormone, a medium chain triglyceride, an endogenous protein, a gene therapy agent, an anti-inflammatory agent (e.g., a non-steroidal anti-inflammatory drug (NSAID)), a vaccine, a vaccine which includes antibodies against a specific protein that is characteristic of a disorder of the subject, a vaccine which includes antibodies against β-amyloid, a vaccine which includes antibodies against tau protein, a combination of a vaccine and an anti-inflammatory drug, a component of a vaccine, and a derivative of a vaccine. Diagnostic agents appropriate for delivery using the techniques described herein include, but are not limited to, an agent for facilitating diagnostic imaging (e.g., a radio-opaque material for facilitating a CT scan), or an antibody.

For some applications, energy applicator 20 is configured or positioned to target energy to an area of the BBB, rather than the entire BBB. For example, when facilitating the delivery of a chemotherapy agent, the energy may be targeted to an area of the BBB in a vicinity of a tumor being treated.

In an embodiment of the present invention, an NO facilitator encapsulated in carrier system 40 is supplied to a body of subject 32, and energy applicator 20 is activated to apply energy to the carrier system so as to cause the carrier system to release the NO facilitator in a vicinity of the BBB, and thereby cause a controlled increase in permeability of CNS blood vessels, vasodilation of CNS blood vessels, and/or an increase in CNS blood flow. (“CNS blood flow” includes both cephalic blood flow and spinal cord blood flow. “Cephalic blood flow” includes cerebral and cerebellar blood flow.) Such increased permeability, vasodilation, and/or CNS blood flow are generally beneficial for treating cerebrovascular disorders such as acute ischemic stroke and cerebral vasospasms after subarachnoid hemorrhage. For example, such treatment may increase survival of neuronal tissue during and following an ischemic event. Such increased permeability, vasodilation, and/or CNS blood flow are also generally beneficial for treating CNS disorders, such as vasospasm of CNS blood vessels, Alzheimer's disease, Parkinson's disease, epilepsy, multiple sclerosis, schizophrenia, depression, stress, obesity, pain, anxiety, and any other CNS disorders that are directly or indirectly affected by changes in CNS blood flow, vasodilation, or BBB permeability changes. For example, increased CNS blood flow may improve brain tissue metabolic state.

In an embodiment of the present invention, an NO facilitator encapsulated in carrier system 40 is supplied to a body of subject 32, and energy applicator 20 is activated to apply energy to the carrier system so as to cause the carrier system to release the NO facilitator in a vicinity of the BBB, and thereby increase clearance of a CNS constituent related to a CNS disorder, from the CNS, through the BBB, and into a systemic blood circulation of the subject. Such increased clearance is considered to be potentially beneficial for treating CNS disorders by lowering the concentration of the CNS-disorder-related constituent in the CNS, which typically reduces the biochemical burden of the constituent. CNS disorders for which this treatment method can be beneficial include, but are not limited to, glaucoma, macular edema, Gaucher's disease, late-onset Tay-Sachs, diabetic retinopathy, vasospasm of CNS blood vessels, Huntington's disease, Alzheimer's disease, Parkinson's disease, epilepsy, multiple sclerosis, ALS, CBD, and other neurodegenerative disorders. Typically, CNS-disorder-related constituents which leave the CNS in response to this treatment include beta-amyloid, PS1, PS2, tau-protein, huntingtin, GM1, GM2, and GM3.

In an embodiment of the present invention, a method for diagnosing a CNS disorder comprises supplying an NO facilitator encapsulated in carrier system 40 to a body of subject 32, and activating energy applicator 20 to apply energy to the carrier system so as to cause the carrier system to release the NO facilitator in a vicinity of the BBB, and thereby increase clearance of a CNS constituent related to the CNS disorder, from the CNS (e.g., the brain, an eye, the spinal cord), through the BBB, and into another body compartment of the subject. For example, the other body compartment may include a systemic blood circulation of the subject, plasma of the subject, serum of the subject, or ascites of the subject. Once in the other body compartment, the CNS constituent is analyzed in order to facilitate a diagnosis of the CNS disorder. CNS disorders for which this treatment method can be beneficial include, but are not limited to, glaucoma, macular edema, Gaucher's disease, late-onset Tay-Sachs, diabetic retinopathy, vasospasm of CNS blood vessels, Huntington's disease, Alzheimer's disease, Parkinson's disease, tumors, epilepsy, multiple sclerosis, ALS, CBD, and other neurodegenerative disorders.

In an embodiment of the present invention, a method for treating a disorder of subject 32 comprises supplying, to a body of subject 32, an NO inhibitor or antagonist encapsulated in carrier system 40, and activating energy applicator 20 to apply energy to the carrier system so as to cause the carrier system to release the NO inhibitor/antagonist in a vicinity of a BBB of the subject, so as to decrease permeability of the BBB, thereby treating the disorder. Such reduced permeability of the BBB may be beneficial for treating or reducing the effects of multiple sclerosis, migraine headaches, neuroinflammation, and damage caused to the BBB by viral, bacterial, fungal, and/or parasitic infections, and/or by bacterial toxins. Delivery of an NO inhibitor/antagonist to brain 30 may also protect tissue from NO damage. Examples of NO inhibitor/antagonists include, but are not limited to, analogs of L-arginine.

In an embodiment of the present invention, a method for treating a disorder of or related to an eye of subject 32 comprises supplying, to a body of subject 32, an NO facilitator or an NO inhibitor/antagonist encapsulated in carrier system 40, positioning energy applicator 20 in a vicinity of the eye, and activating the energy applicator to apply energy to the carrier system so as to cause the carrier system to release the NO facilitator or NO inhibitor/antagonist in a vicinity of the eye, so as to increase or decrease permeability of the BBB in the vicinity of the eye, thereby treating the disorder. Because the eye is an extracranial element of the CNS, energy applied by energy applicator 20, such as ultrasound energy, generally achieves greater penetration of ocular structures than of other regions of the CNS, at a given power level. Energy applicator 20 therefore typically applies less energy to the eye than to other regions of the CNS.

In an embodiment of the present invention, a method for treating a disorder of the CNS of subject 32 comprises supplying, to a body of subject 32, a light-activated NO precursor, and applying light to the NO precursor so as to cause the release of NO in a vicinity of the BBB, and thereby increase permeability of the BBB, so as to treat the disorder. Several NO precursors are activated by light at a specific wavelength, e.g., nitrosothiols, organic nitrites, N-nitrosamines, and nitrosimines (see the above-mentioned article by Wang et al.). For some applications, energy applicator 20 is configured or positioned to apply the light through an eye of subject 32, in order to increase the permeability of the BBB in a vicinity of the eye (which is sometimes referred to as the blood-retinal barrier). Alternatively, natural light entering the eye is used to activate the NO precursor, in which case use of energy applicator 20 is optional. Alternatively, energy applicator 20 is configured or positioned to apply the light in a vicinity of blood vessel 42, either externally or using a catheter, as described hereinbelow, in order to increase the permeability of the BBB in a wider area of brain 30.

For some applications, the light-activated NO precursor is encapsulated in carrier system 40. Carrier system 40 is either (a) light-activated, in which case the applied light both releases the NO precursor and activates the NO precursor, or (b) activated by another energy type, such as ultrasound energy.

In an embodiment of the present invention, a method for treating pain, such as lower-back pain, of subject 32 comprises supplying, to the body of subject 32, an NO facilitator encapsulated in carrier system 40, and an analgesic. Energy applicator 20, which typically, but not necessarily, comprises an ultrasound transducer in this embodiment, is activated to apply energy to carrier system 40 so as to cause the carrier system to release the NO facilitator. The NO facilitator releases NO in a vicinity of the BBB, so as to increase passage of the analgesic from the blood circulation of subject 32, through the BBB, and into the CNS of the subject (the brain or the spinal cord). For some applications, the NO facilitator is encapsulated with the analgesic, such as in a pill for oral administration. Alternatively, the NO facilitator is not encapsulated with the analgesic, and the NO facilitator and the analgesic are administered separately. For some applications, subject 32 self-administers the NO facilitator and the analgesic, such as at home, typically under the direction of a healthcare worker. Administration of the analgesic using these techniques may allow the use of lower total dosages of the analgesic, while achieving the same or greater levels of pain relief as achieved using conventional analgesic administration.

In an embodiment of the present invention, energy applicator 20 is positioned in a vicinity of a lower back of subject 32, such that application of the energy causes the analgesic to locally permeate the BBB into a spinal cord of the subject. For some applications, the energy applicator is incorporated into a chair. The subject sits in the chair during or soon after administration of the NO facilitator and the analgesic, and activates the energy applicator, such as by pressing a button, in order to attain pain relief. Alternatively, energy applicator 20 is incorporated in another device that positions the energy applicator at the desired location. For example, the device may be a belt, e.g., similar to a weight lifter's belt. Optionally, the energy applicator is preprogrammed and/or programmable by a healthcare worker to set a maximum frequency of use (e.g., number of times per day) and/or maximum duration of use (e.g., minutes per 24-hour period).

For some applications, energy applicator 20 is incorporated in a catheter, which is adapted to be inserted in a blood vessel of subject 32 and manipulated into the vicinity of blood vessel 42 or brain 30, as appropriate. For example, techniques described in the above-mentioned US Patent Application Publication 2003/0092667 may be utilized, mutatis mutandis.

Although the techniques described herein have sometimes been described with respect to brain 30, these techniques are also generally appropriate for a spinal cord of subject 32. For example, passage through the BBB may be facilitated in a vicinity of the spinal cord.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method for delivering molecules to a central nervous system (CNS) of a subject, the method comprising:
supplying the molecules to a blood circulation of the CNS;
supplying, to a body of the subject, a carrier system that encapsulates a nitric oxide (NO) facilitator; and
applying energy to the carrier system at an energy level sufficient to cause the carrier system to release the NO facilitator in a blood circulation of the subject in a vicinity of a blood-brain barrier (BBB) of the subject and thereby increase passage of the molecules from the blood circulation of the CNS, through the BBB, and into the CNS of the subject.

2. The method according to claim 1, wherein supplying the molecules to the blood circulation of the CNS comprises supplying the molecules to a blood circulation of a brain of the subject.

3. The method according to claim 1, wherein supplying the molecules to the blood circulation of the CNS comprises supplying the molecules to a blood circulation of a spinal cord of the subject.

4. The method according to claim 1, wherein supplying the molecules comprises administering the molecules to a systemic blood circulation of the subject.

5. The method according to claim 1, wherein the energy is selected from the list consisting of: microwave energy, radiofrequency energy, and magnetic induction oscillating energy, and wherein applying the energy comprises applying the selected energy.

6. The method according to claim 1, wherein the energy includes light energy, and wherein applying the energy comprises applying the light energy.

7. The method according to claim 1, wherein the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers, and wherein supplying the carrier system comprises supplying the selected carrier system.

8. The method according to claim 1, wherein the carrier system is selected from the list consisting of: a cell, a cell ghost, a lipoprotein, and a liposome, and wherein supplying the carrier system comprises supplying the selected carrier system.

9. The method according to claim 1, wherein supplying the molecules comprises selecting molecules effective in treating a condition selected from the list consisting of: an ischemic condition, vasospasm of a blood vessel of the CNS, infection, a CNS condition, a primary tumor of the CNS, and metastases in the CNS.

10. The method according to claim 1, wherein supplying the molecules comprises selecting molecules effective in treating a condition selected from the list consisting of: pain and lower-back pain.

11. The method according to claim 1, wherein supplying the molecules comprises selecting molecules effective in treating a condition selected from the list consisting of: Alzheimer's disease, Parkinson's disease, epilepsy, multiple sclerosis, Lou Gehrig's Disease (ALS), corticobasal degeneration (CBD), and a neurodegenerative disorder.

12. The method according to claim 1, wherein applying the energy comprises applying the energy from an energy applicator incorporated in a chair, in which the subject sits.

13. The method according to claim 1, wherein applying the energy comprises applying the energy from an energy applicator incorporated in a belt, which the subject wears.

14. The method according to claim 1, wherein the energy includes ultrasound energy, and wherein applying the energy comprises applying the ultrasound energy.

15. The method according to claim 1, wherein applying the energy comprises applying the energy to a back of the subject.

16. The method according to claim 1, wherein supplying the molecules comprises selecting molecules effective in treating a condition of an eye of the subject.

17. The method according to claim 16, wherein the energy includes ultrasound energy, and wherein applying the energy comprises applying the ultrasound energy.

18. The method according to claim 1, wherein supplying the molecules comprises selecting molecules effective in treating a condition of an ear of the subject.

19. The method according to claim 18, wherein the energy includes ultrasound energy, and wherein applying the energy comprises applying the ultrasound energy.

20. The method according to claim 1, wherein the molecules include a pharmaceutical agent, and wherein supplying the molecules comprises supplying the pharmaceutical agent.

21. The method according to claim 20, wherein the pharmaceutical agent includes an analgesic, and wherein supplying the pharmaceutical agent comprises supplying the analgesic.

22. The method according to claim 20, wherein the pharmaceutical agent is selected from the list consisting of: a neuroprotective agent, an enzyme, a chemotherapy agent, a virus that is a vector of gene therapy, an antiviral agent, an antibacterial agent, a glutamate receptor antagonist, an NMDA receptor blocker, a cholinesterase inhibitor, an agent having an inhibitory effect on derivation of β-amyloid from amyloid precursor protein, a β-amyloid inhibitor, an inhibitor of protein tyrosine phosphatases, a stimulant of nerve regeneration, a nerve growth factor, a compound that stimulates production of nerve growth factor, a microglial activation modulator, an antioxidant, a hormone, a medium chain triglyceride, an endogenous protein, a gene therapy agent, an anti-inflammatory agent, a non-steroidal anti-inflammatory drug (NSAID), a vaccine, a vaccine which includes antibodies against a specific protein that is characteristic of a disorder of the subject, a vaccine which includes antibodies against β-amyloid, a vaccine which includes antibodies against tau protein, a combination of a vaccine and an anti-inflammatory drug, a component of a vaccine, and a derivative of a vaccine, and wherein supplying the pharmaceutical agent comprises supplying the selected pharmaceutical agent.

23. The method according to claim 1, wherein the molecules include a diagnostic agent, and wherein supplying the molecules comprises supplying the diagnostic agent.

24. The method according to claim 23, wherein the diagnostic agent includes an agent for facilitating diagnostic imaging, and wherein supplying the diagnostic agent comprises supplying the agent for facilitating diagnostic imaging.

25. The method according to claim 23, wherein the diagnostic agent includes an antibody, and wherein supplying the diagnostic agent comprises supplying the antibody.

26. The method according to claim 1, wherein the molecules are encapsulated in the carrier system, and wherein supplying the molecules comprises supplying the carrier system to the body.

27. The method according to claim 26, wherein the molecules are mixed with the NO facilitator, and wherein supplying the molecules comprises supplying the carrier system to the body.

28. The method according to claim 26, wherein the molecules are chemically conjugated with the NO facilitator, and wherein supplying the molecules comprises supplying the carrier system to the body.

29. The method according to claim 1, wherein supplying the carrier system comprises implanting the carrier system in a vicinity of a blood vessel of an upper circulation of the subject, which blood vessel supplies blood to a brain of the subject.

30. The method according to claim 29, wherein implanting the carrier system comprises implanting the carrier system in an artery of the subject selected from the list consisting of: a carotid artery of the subject and a vertebral artery of the subject.

31. The method according to claim 1, wherein supplying the carrier system comprises administering the carrier system to a systemic blood circulation of the subject.

32. The method according to claim 31, wherein applying the energy comprises applying the energy in a vicinity of an eye of the subject.

33. The method according to claim 32, wherein the energy includes light energy, and wherein applying the energy comprises applying the light energy.

34. The method according to claim 32, wherein the energy includes ultrasound energy, and wherein applying the energy comprises applying the ultrasound energy.

35. The method according to claim 32, wherein applying the energy comprises exposing the subject to ambient light.

36. The method according to claim 31, wherein applying the energy comprises applying the energy to substantially an entire brain of the subject.

37. The method according to claim 31, wherein applying the energy comprises targeting the energy to a specific area of a brain of the subject.

38. The method according to claim 37, wherein targeting the energy comprises targeting the energy to an area of the BBB in a vicinity of a tumor.

39. A method for treating a central nervous system (CNS) disorder of a subject, the method comprising:
supplying, to a body of the subject, a carrier system that encapsulates a nitric oxide (NO) facilitator; and
applying energy to the carrier system at an energy level sufficient to cause the carrier system to release the NO facilitator in a blood circulation of the subject in a vicinity of a CNS of the subject and thereby cause vasodilation of CNS brain blood vessels and an increase in CNS blood flow, so as to treat the CNS disorder.

40. The method according to claim 39, wherein applying the energy comprises configuring the application of the energy to cause the carrier system to release the NO facilitator in the blood circulation in a vicinity of a brain of the subject.

41. The method according to claim 39, wherein applying the energy comprises configuring the application of the energy to cause the carrier system to release the NO facilitator in the blood circulation in a vicinity of a spinal cord of the subject.

42. The method according to claim 39, wherein the CNS disorder includes a disorder of an eye of the subject, and wherein applying the energy comprises applying the energy so as to treat the eye disorder.

43. The method according to claim 39, wherein the CNS disorder includes a disorder of an ear of the subject, and wherein applying the energy comprises applying the energy so as to treat the ear disorder.

44. The method according to claim 39, wherein the energy is selected from the list consisting of: microwave energy, radiofrequency energy, and magnetic induction oscillating energy, and wherein applying the energy comprises applying the selected energy.

45. The method according to claim 39, wherein the energy includes light energy, and wherein applying the energy comprises applying the light energy.

46. The method according to claim 39, wherein the energy includes ultrasound energy, and wherein applying the energy comprises applying the ultrasound energy.

47. The method according to claim 39, wherein the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers, and wherein supplying the carrier system comprises supplying the selected carrier system.

48. The method according to claim 39, wherein the carrier system is selected from the list consisting of: a cell, a cell ghost, a lipoprotein, and a liposome, and wherein supplying the carrier system comprises supplying the selected carrier system.

49. The method according to claim 39, wherein the CNS disorder includes a disorder of the brain of the subject, and wherein applying the energy comprises applying the energy so as to treat the brain disorder.

50. The method according to claim 39, wherein the CNS disorder is selected from the list consisting of: vasospasm of a blood vessel of the CNS, Gaucher's disease, late-onset Tay-Sachs, Huntington's disease, Alzheimer's disease, Parkinson's disease, epilepsy, multiple sclerosis, and schizophrenia, and wherein applying the energy comprises applying the energy so as to treat the selected CNS disorder.

51. The method according to claim 39, wherein the CNS disorder is selected from the list consisting of: glaucoma, macular edema, and diabetic retinopathy, and wherein applying the energy comprises applying the energy so as to treat the selected CNS disorder.

52. The method according to claim 39, wherein the CNS disorder is selected from the list consisting of: depression, stress, obesity, pain, and anxiety, and wherein applying the energy comprises applying the energy so as to treat the selected CNS disorder.

53. The method according to claim 39, wherein supplying the carrier system comprises administering the carrier system to a systemic blood circulation of the subject.

54. The method according to claim 39, wherein the CNS disorder includes a vascular disorder of the CNS, and wherein applying the energy comprises applying the energy so as to treat the CNS vascular disorder.

55. The method according to claim 54, wherein the CNS vascular disorder includes cerebral vasospasms after subarachnoid hemorrhage of the subject, and wherein applying the energy comprises applying the energy so as to treat the cerebral vasospasms.

56. The method according to claim 39, wherein supplying the carrier system comprises implanting the carrier system in a vicinity of a blood vessel of an upper circulation of the subject, which blood vessel supplies blood to a brain of the subject.

57. The method according to claim 56, wherein implanting the carrier system comprises implanting the carrier system in an artery of the subject selected from the list consisting of: a carotid artery of the subject and a vertebral artery of the subject.

58. The method according to claim 39, wherein the CNS disorder includes an ischemic disorder of the subject, and wherein applying the energy comprises applying the energy at a level sufficient to cause vasodilation and thereby treat the ischemic disorder.

59. The method according to claim 58, wherein the ischemic disorder is selected from the list consisting of: arterial vein occlusion and vein thrombosis, and wherein applying the energy comprises applying the energy so as to treat the selected ischemic disorder.

60. The method according to claim 58, wherein the ischemic disorder includes retinal vein occlusion, and wherein applying the energy comprises applying the energy so as to treat the retinal vein occlusion.

61. The method according to claim 58, wherein the ischemic disorder includes a chronic ischemic disorder of the subject, and wherein applying the energy comprises applying the energy so as to treat the chronic ischemic disorder.

62. The method according to claim 58, wherein the ischemic disorder includes an acute ischemic event of the subject, and wherein applying the energy comprises applying the energy so as to treat the acute ischemic event.

63. The method according to claim 62, wherein the acute ischemic event includes acute ischemic stroke of the subject, and wherein applying the energy comprises applying the energy so as to treat the acute ischemic stroke.

64. A method for treating a disorder of a central nervous system (CNS) of a subject, the method comprising:
supplying, to a body of the subject, a carrier system encapsulating a nitric oxide (NO) facilitator; and
applying energy to the carrier system at an energy level sufficient to cause the carrier system to release the NO facilitator in a blood circulation of the subject in a vicinity of a blood-brain barrier (BBB) of the subject and thereby increase clearance of a CNS constituent related to the CNS disorder, from the CNS, through the BBB, and into a systemic blood circulation of the subject, so as to treat the CNS disorder.

65. The method according to claim 64, wherein applying the energy comprises configuring the energy to induce vasodilation, and thereby increase the clearance of the CNS constituent, to an extent that decreases edema of a brain of the subject.

66. The method according to claim 64, wherein applying the energy comprises configuring the energy to induce vasodilation, and thereby increase the clearance of the CNS constituent, to an extent that decreases edema of an eye of the subject.

67. The method according to claim 64, wherein applying the energy comprises configuring the energy to increase the clearance of the CNS constituent from a brain of the subject, through the BBB, and into the systemic blood circulation.

68. The method according to claim 64, wherein applying the energy comprises configuring the energy to increase the clearance of the CNS constituent from an eye of the subject, through the BBB, and into the systemic blood circulation.

69. The method according to claim 64, wherein applying the energy comprises configuring the energy to increase the clearance of the CNS constituent from a spinal cord of the subject, through the BBB, and into the systemic blood circulation.

70. The method according to claim 64, wherein the CNS disorder is selected from the list consisting of: Gaucher's disease, late-onset Tay-Sachs, vasospasm of a blood vessel of the CNS, Huntington's disease, Alzheimer's disease, Parkinson's disease, epilepsy, multiple sclerosis, Lou Gehrig's Disease (ALS), and corticobasal degeneration (CBD), and wherein applying the energy comprises applying the energy so as to treat the selected CNS disorder.

71. The method according to claim 64, wherein the CNS disorder is selected from the list consisting of: glaucoma, macular edema, and diabetic retinopathy, and wherein applying the energy comprises applying the energy so as to treat the selected CNS disorder.

72. The method according to claim 64, wherein the energy is selected from the list consisting of: microwave energy, radiofrequency energy, and magnetic induction oscillating energy, and wherein applying the energy comprises applying the selected energy.

73. The method according to claim 64, wherein the energy includes ultrasound energy, and wherein applying the energy comprises applying the ultrasound energy.

74. The method according to claim 64, wherein the energy includes light energy, and wherein applying the energy comprises applying the light energy.

75. The method according to claim 64, wherein the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers, and wherein supplying the carrier system comprises supplying the selected carrier system.

76. The method according to claim 64, wherein the carrier system is selected from the list consisting of: a cell, a cell ghost, a lipoprotein, and a liposome, and wherein supplying the carrier system comprises supplying the selected carrier system.

77. The method according to claim 64, wherein supplying the carrier system comprises administering the carrier system to a systemic blood circulation of the subject.

78. The method according to claim 64, wherein supplying the carrier system comprises implanting the carrier system in a vicinity of a blood vessel of an upper circulation of the subject, which blood vessel supplies blood to a brain of the subject.

79. The method according to claim 78, wherein implanting the carrier system comprises implanting the carrier system in an artery of the subject selected from the list consisting of: a carotid artery of the subject and a vertebral artery of the subject.

80. A method for facilitating a diagnosis of a disorder of a central nervous system (CNS) of a subject, the method comprising:
supplying, to a body of the subject, a carrier system encapsulating a nitric oxide (NO) facilitator; and
applying energy to the carrier system at an energy level sufficient to cause the carrier system to release the NO facilitator in a blood circulation of the subject in a vicinity of a blood-brain barrier (BBB) of the subject and thereby increase clearance of a CNS constituent related to the CNS disorder, from the CNS, through the BBB, and into another body compartment of the subject, so as to facilitate the diagnosis of the CNS disorder.

81. The method according to claim 80, wherein applying the energy comprises configuring the energy to increase the clearance of the CNS constituent from a brain of the subject, through the BBB, and into the other body compartment.

82. The method according to claim 80, wherein applying the energy comprises configuring the energy to increase the clearance of the CNS constituent from an eye of the subject, through the BBB, and into the other body compartment.

83. The method according to claim 80, wherein applying the energy comprises configuring the energy to increase the clearance of the CNS constituent from a spinal cord of the subject, through the BBB, and into the other body compartment.

84. The method according to claim 80, wherein the other body compartment includes a systemic blood circulation of the subject, and wherein applying the energy comprises setting the energy level to be sufficient to increase the clearance of the CNS constituent from the CNS to the systemic blood circulation.

85. The method according to claim 80, wherein the other body compartment includes plasma of the subject, and wherein applying the energy comprises setting the energy level to be sufficient to increase the clearance of the CNS constituent from the CNS to the plasma.

86. The method according to claim 80, wherein the other body compartment includes serum of the subject, and wherein applying the energy comprises setting the energy level to be sufficient to increase the clearance of the CNS constituent from the CNS to the serum.

87. The method according to claim 80, wherein the other body compartment is ascites of the subject, and wherein applying the energy comprises setting the energy level to be sufficient to increase the clearance of the CNS constituent from the CNS to the ascites.

88. The method according to claim 80, wherein the CNS disorder is selected from the list consisting of: Gaucher's disease, late-onset Tay-Sachs, vasospasm of a blood vessel of the CNS, Huntington's disease, Alzheimer's disease, Parkinson's disease, a tumor, epilepsy, multiple sclerosis, Lou Gehrig's Disease (ALS), and corticobasal degeneration (CBD).

89. The method according to claim 80, wherein the CNS disorder is selected from the list consisting of: glaucoma, macular edema, and diabetic retinopathy.

90. The method according to claim 80, wherein the energy is selected from the list consisting of: microwave energy, radiofrequency energy, and magnetic induction oscillating energy, and wherein applying the energy comprises applying the selected energy.

91. The method according to claim 80, wherein the energy includes ultrasound energy, and wherein applying the energy comprises applying the ultrasound energy.

92. The method according to claim 80, wherein the energy includes light energy, and wherein applying the energy comprises applying the light energy.

93. The method according to claim 80, wherein the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers, and wherein supplying the carrier system comprises supplying the selected carrier system.

94. The method according to claim 80, wherein the carrier system is selected from the list consisting of: a cell, a cell ghost, a lipoprotein, and a liposome, and wherein supplying the carrier system comprises supplying the selected carrier system.

95. The method according to claim 80, wherein supplying the carrier system comprises administering the carrier system to a systemic blood circulation of the subject.

96. The method according to claim 80, wherein supplying the carrier system comprises implanting the carrier system in a vicinity of a blood vessel of an upper circulation of the subject, which blood vessel supplies blood to a brain of the subject.

97. The method according to claim 96, wherein implanting the carrier system comprises implanting the carrier system in an artery of the subject selected from the list consisting of: a carotid artery of the subject and a vertebral artery of the subject.

98. A method for treating a disorder of a subject, the method comprising:
supplying, to a body of the subject, a carrier system encapsulating a nitric oxide (NO) inhibitor; and
applying energy to the carrier system at an energy level sufficient to cause the carrier system to release the NO inhibitor in a blood circulation of the subject in a vicinity of a blood-brain barrier (BBB) of the subject and thereby decrease permeability of the BBB, so as to treat the disorder.

99. The method according to claim 98, wherein the disorder includes multiple sclerosis, and wherein applying the energy comprises applying the energy so as to treat the multiple sclerosis.

100. The method according to claim 98, wherein the disorder includes migraine headache, and wherein applying the energy comprises applying the energy so as to treat the migraine headache.

101. The method according to claim 98, wherein the disorder includes neuroinflammation, and wherein applying the energy comprises applying the energy so as to treat the neuroinflammation.

102. The method according to claim 98, wherein the disorder includes damage caused to the BBB by infection, and wherein applying the energy comprises applying the energy so as to treat the damage.

103. The method according to claim 98, wherein the disorder includes damage caused to the BBB by a bacterial toxin, and wherein applying the energy comprises applying the energy so as to treat the damage.

104. The method according to claim 98, wherein the energy is selected from the list consisting of: microwave energy, radiofrequency energy, and magnetic induction oscillating energy, and wherein applying the energy comprises applying the selected energy.

105. The method according to claim 98, wherein the energy includes ultrasound energy, and wherein applying the energy comprises applying the ultrasound energy.

106. The method according to claim 98, wherein the energy includes light energy, and wherein applying the energy comprises applying the light energy.

107. The method according to claim 98, wherein the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers, and wherein supplying the carrier system comprises supplying the selected carrier system.

108. The method according to claim 98, wherein the carrier system is selected from the list consisting of: a cell, a cell ghost, a lipoprotein, and a liposome, and wherein supplying the carrier system comprises supplying the selected carrier system.

109. The method according to claim 98, wherein supplying the carrier system comprises implanting the carrier system in a vicinity of a blood vessel of an upper circulation of the subject, which blood vessel supplies blood to a brain of the subject.

110. The method according to claim 109, wherein implanting the carrier system comprises implanting the carrier system in an artery of the subject selected from the list consisting of: a carotid artery of the subject and a vertebral artery of the subject.

111. The method according to claim 98, wherein supplying the carrier system comprises administering the carrier system to a systemic blood circulation of the subject.

112. The method according to claim 111, wherein applying the energy comprises applying the energy to a vicinity of an eye of the subject.

113. A method for treating a disorder of a subject, the method comprising:
supplying, to the blood circulation of a brain of the subject, a light-activated nitric oxide (NO) precursor; and
applying light to the NO precursor at a level sufficient to cause the NO precursor to release NO in a vicinity of a blood-brain barrier (BBB) of the subject and thereby increase permeability of the BBB, so as to treat the disorder.

114. The method according to claim 113, comprising supplying molecules to the blood circulation of the brain, wherein applying the light comprises configuring the light applied to the NO precursor to be of a level sufficient to cause the NO precursor to release the NO and thereby increase passage of the molecules from the blood circulation of the brain, through the BBB, and into the CNS of the subject.

115. The method according to claim 113, wherein applying the light comprises applying the light through an eye of the subject.

116. The method according to claim 113, wherein the NO precursor is selected from the list consisting of: a nitrosothiol, an organic nitrite, an N-nitrosamine, and a nitrosamine, and wherein supplying the NO precursor comprises supplying the selected NO precursor.

117. The method according to claim 113, wherein the disorder includes a disorder of an eye of the subject, and wherein applying the light comprises applying the light so as to treat the eye disorder.

118. The method according to claim 113, wherein the disorder includes a disorder of an ear of the subject, and wherein applying the light comprises applying the light so as to treat the ear disorder.

119. The method according to claim 113, comprising:
supplying, to a body of the subject, a carrier system encapsulating the light-activated NO precursor; and
applying energy to the carrier system at an energy level sufficient to cause the carrier system to release the light-activated NO facilitator in a blood circulation of the subject in a vicinity of the BBB.

120. The method according to claim 119, wherein applying the energy comprises applying the light.

121. The method according to claim 119, wherein the energy is selected from the list consisting of: microwave energy, radiofrequency energy, and magnetic induction oscillating energy, and wherein applying the energy comprises applying the selected energy.

122. The method according to claim 119, wherein the energy includes ultrasound energy, and wherein applying the energy comprises applying the ultrasound energy.

123. The method according to claim 119, wherein supplying the carrier system comprises implanting the carrier system in a vicinity of a blood vessel of an upper circulation of the subject, which blood vessel supplies blood to the brain.

124. The method according to claim 123, wherein implanting the carrier system comprises implanting the carrier system in an artery of the subject selected from the list consisting of: a carotid artery of the subject and a vertebral artery of the subject.

125. The method according to claim 119, wherein supplying the carrier system comprises administering the carrier system to a systemic blood circulation of the subject.

126. The method according to claim 125, wherein applying the energy comprises applying the energy in a vicinity of the eye.

127. A method for treating a disorder of a subject, comprising:
supplying, to a body of the subject, a carrier system encapsulating a nitric oxide (NO) facilitator; and
applying energy to the carrier system at an energy level sufficient to cause the carrier system to release the NO facilitator in a blood circulation of the subject in a vicinity of a blood-brain barrier (BBB) of the subject and thereby increase passage of a substance through the BBB between a spinal cord of the subject and the blood circulation, so as to treat the disorder.

128. The method according to claim 127, wherein the energy is selected from the list consisting of: microwave energy, radiofrequency energy, and magnetic induction oscillating energy, and wherein applying the energy comprises applying the selected energy.

129. The method according to claim 127, wherein the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers, and wherein supplying the carrier system comprises supplying the selected carrier system.

130. The method according to claim 127, wherein the carrier system is selected from the list consisting of: a cell, a cell ghost, a lipoprotein, and a liposome, and wherein supplying the carrier system comprises supplying the selected carrier system.

131. The method according to claim 127, wherein applying the energy comprises applying the energy from an energy applicator incorporated in a chair, in which the subject sits.

132. The method according to claim 127, wherein applying the energy comprises applying the energy from an energy applicator incorporated in a belt, which the subject wears.

133. The method according to claim 127, wherein the substance includes a constituent of the spinal cord, and wherein applying the energy comprises configuring the energy to increase clearance of the constituent from the spinal cord, through the BBB, to the blood circulation.

134. The method according to claim 127, wherein the energy includes ultrasound energy, and wherein applying the energy comprises applying the ultrasound energy.

135. The method according to claim 127, wherein the energy includes light energy, and wherein applying the energy comprises applying the light energy.

136. The method according to claim 127, wherein applying the energy comprises applying the energy to a back of the subject.

137. The method according to claim 127, wherein the substance comprises molecules, and comprising supplying the molecules to the blood circulation, wherein applying the energy comprises configuring the energy to increase passage of the molecules from the blood circulation, through the BBB, to the spinal cord.

138. The method according to claim 137, wherein the molecules include a diagnostic agent, and wherein supplying the molecules comprises supplying the diagnostic agent.

139. The method according to claim 137, wherein the molecules include a pharmaceutical agent, and wherein supplying the molecules comprises supplying the pharmaceutical agent.

140. The method according to claim 139, wherein the pharmaceutical agent includes an analgesic, and wherein supplying the pharmaceutical agent comprises supplying the analgesic.

141. The method according to claim 137, wherein the molecules are encapsulated in the carrier system, and wherein supplying the molecules comprises supplying the carrier system to the body.

142. The method according to claim 141, wherein the molecules are mixed with the NO facilitator, and wherein supplying the molecules comprises supplying the carrier system to the body.

143. The method according to claim 141, wherein the molecules are chemically conjugated with the NO facilitator, and wherein supplying the molecules comprises supplying the carrier system to the body.

144. A method for facilitating a diagnosis of a disorder of a subject, comprising:
supplying, to a body of the subject, a carrier system encapsulating a nitric oxide (NO) facilitator; and
applying energy to the carrier system at an energy level sufficient to cause the carrier system to release the NO facilitator in a blood circulation of the subject in a vicinity of a blood-brain barrier (BBB) of the subject and thereby increase passage of a spinal cord constituent, from a spinal cord of the subject, through the BBB, and into a systemic blood circulation of the subject, so as to facilitate the diagnosis of the disorder.

145. A molecule delivery system comprising:
molecules adapted to be supplied to a blood circulation of a central nervous system (CNS) of a subject;
a carrier system adapted to be supplied to a body of the subject, the carrier system encapsulating a nitric oxide (NO) facilitator; and
a transducer, adapted to apply ultrasound energy to the carrier system at an energy level sufficient to cause the carrier system to release the NO facilitator in a blood circulation of the subject in a vicinity of a blood-brain barrier (BBB) of the subject and thereby increase passage of the molecules from the blood circulation of the CNS, through the BBB, and into the CNS of the subject.

146. The molecule delivery system according to claim 145, wherein the transducer is adapted to apply the energy to a back of the subject.

147. The molecule delivery system according to claim 145, wherein the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers.

148. The molecule delivery system according to claim 145, wherein the carrier system is selected from the list consisting of: a cell, a cell ghost, a lipoprotein, and a liposome.

149. The molecule delivery system according to claim 145, wherein the molecules comprise molecules effective in treating a condition selected from the list consisting of: an ischemic condition, vasospasm of a blood vessel of the CNS, infection, a CNS condition, a primary tumor of the CNS, and metastases in the CNS.

150. The molecule delivery system according to claim 145, wherein the molecules comprise molecules effective in treating a condition selected from the list consisting of: pain and lower-back pain.

151. The molecule delivery system according to claim 145, wherein the molecules comprise molecules effective in treating a condition selected from the list consisting of: Alzheimer's disease, Parkinson's disease, epilepsy, multiple sclerosis, Lou Gehrig's Disease (ALS), corticobasal degeneration (CBD), and a neurodegenerative disorder.

152. The molecule delivery system according to claim 145, wherein the molecules comprise molecules effective in treating a condition of an eye of the subject.

153. The molecule delivery system according to claim 145, wherein the molecules comprise molecules effective in treating a condition of an ear of the subject.

154. The molecule delivery system according to claim 145, comprising a chair, in which the transducer is incorporated, and which is adapted to be sat in by the subject.

155. The molecule delivery system according to claim 145, comprising a belt, in which the transducer is incorporated, and which is adapted to be worn by the subject.

156. The molecule delivery system according to claim 145, wherein the molecules comprise a pharmaceutical agent.

157. The molecule delivery system according to claim 156, wherein the pharmaceutical agent comprises an analgesic.

158. The molecule delivery system according to claim 156, wherein the pharmaceutical agent is selected from the list consisting of: an analgesic agent, a neuroprotective agent, an enzyme, a chemotherapy agent, a virus that is a vector of gene therapy, an antiviral agent, an antibacterial agent, a glutamate receptor antagonist, an NMDA receptor blocker, a cholinesterase inhibitor, an agent having an inhibitory effect on derivation of β-amyloid from amyloid precursor protein, a β-amyloid inhibitor, an inhibitor of protein tyrosine phosphatases, a stimulant of nerve regeneration, a nerve growth factor, a compound that stimulates production of nerve growth factor, a microglial activation modulator, an antioxidant, a hormone, a medium chain triglyceride, an endogenous protein, a gene therapy agent, an anti-inflammatory agent, a non-steroidal anti-inflammatory drug (NSAID), a vaccine, a vaccine which includes antibodies against a specific protein that is characteristic of a disorder of the subject, a vaccine which includes antibodies against β-amyloid, a vaccine which includes antibodies against tau protein, a combination of a vaccine and an anti-inflammatory drug, a component of a vaccine, and a derivative of a vaccine.

159. The molecule delivery system according to claim 145, wherein the molecules comprise a diagnostic agent.

160. The molecule delivery system according to claim 159, wherein the diagnostic agent includes an agent for facilitating diagnostic imaging.

161. The molecule delivery system according to claim 159, wherein the diagnostic agent includes an antibody.

162. The molecule delivery system according to claim 145, wherein the molecules are encapsulated in the carrier system.

163. The molecule delivery system according to claim 162, wherein the molecules are mixed with the NO facilitator.

164. The molecule delivery system according to claim 162, wherein the molecules are chemically conjugated with the NO facilitator.

165. The molecule delivery system according to claim 145, wherein the carrier system is adapted to be implanted in a vicinity of a blood vessel of an upper circulation of the subject, which blood vessel supplies blood to a brain of the subject.

166. The molecule delivery system according to claim 165, wherein the carrier system is adapted to be implanted in an artery of the subject selected from the list consisting of: a carotid artery of the subject and a vertebral artery of the subject.

167. The molecule delivery system according to claim 145, wherein the carrier system is adapted to be administered to a systemic blood circulation of the subject.

168. The molecule delivery system according to claim 167, wherein the transducer is adapted to apply the energy in a vicinity of an eye of the subject.

169. A treatment system for treating a central nervous system (CNS) disorder of a subject, the treatment system comprising:
a carrier system adapted to be supplied to a body of the subject, the carrier system encapsulating a nitric oxide (NO) facilitator; and
a transducer, adapted to apply ultrasound energy to the carrier system at an energy level sufficient to cause the carrier system to release the NO facilitator in a blood circulation of the subject in a vicinity of a blood-brain barrier (BBB) of the subject and thereby cause vasodilation of CNS blood vessels and an increase in CNS blood flow, so as to treat the CNS disorder.

170. The treatment system according to claim 169, wherein the CNS disorder includes a disorder of an eye of the subject, and wherein the transducer is configured to apply the energy so as to treat the eye disorder.

171. The treatment system according to claim 169, wherein the CNS disorder includes a disorder of an ear of the subject, and wherein the transducer is configured to apply the energy so as to treat the ear disorder.

172. The treatment system according to claim 169, wherein the carrier system is selected from the list consisting of: a polymer, an ultrasound-sensitive bio-polymer, a nano-particle cell, a micro-particle, a micelle, an ultrasound-sensitive stabilized pluronic micelle, a microbubble, a microsphere, and a microparticle made of insoluble or biodegradable natural or synthetic polymers.

173. The treatment system according to claim 169, wherein the carrier system is selected from the list consisting of: a cell, a cell ghost, a lipoprotein, and a liposome.

174. The treatment system according to claim 169, wherein the CNS disorder includes a disorder of a brain of the subject, and wherein the transducer is configured to apply the energy so as to treat the brain disorder.

175. The treatment system according to claim 169, wherein the CNS disorder is selected from the list consisting of: vasospasm of a blood vessel of the CNS, Gaucher's disease, late-onset Tay-Sachs, Huntington's disease, Alzheimer's disease, Parkinson's disease, epilepsy, multiple sclerosis, and schizophrenia, and wherein the transducer is configured to apply the energy so as to treat the selected CNS disorder.

176. The treatment system according to claim 169, wherein the CNS disorder is selected from the list consisting of: glaucoma, macular edema, and diabetic retinopathy, and wherein the transducer is configured to apply the energy so as to treat the selected CNS disorder.

177. The treatment system according to claim 169, wherein the CNS disorder is selected from the list consisting of: depression, stress, obesity, pain, and anxiety, and wherein the transducer is configured to apply the energy so as to treat the selected CNS disorder.

178. The treatment system according to claim 169, wherein the carrier system is adapted to be administered to a systemic blood circulation of the subject.

179. The treatment system according to claim 169, wherein the CNS disorder includes a vascular disorder of the CNS, and wherein the transducer is configured to apply the energy so as to treat the CNS vascular disorder.

180. The treatment system according to claim 179, wherein the CNS vascular disorder includes cerebral vasospasms after subarachnoid hemorrhage of the subject, and wherein the transducer is configured to apply the energy so as to treat the cerebral vasospasms.

181. The treatment system according to claim 169, wherein the carrier system is adapted to be implanted in a vicinity of a blood vessel of an upper circulation of the subject, which blood vessel supplies blood to a brain of the subject.

182. The treatment system according to claim 181, wherein the carrier system is adapted to be implanted in an artery of the subject selected from the list consisting of: a carotid artery of the subject and a vertebral artery of the subject.

183. The treatment system according to claim 169, wherein the CNS disorder includes an ischemic disorder of the subject, and wherein the transducer is configured to apply the energy at a level sufficient to cause vasodilation and thereby treat the ischemic disorder.

184. The treatment system according to claim 183, wherein the ischemic disorder is selected from the list consisting of: arterial vein occlusion and vein thrombosis, and wherein the transducer is configured to apply the energy so as to treat the selected ischemic disorder.

185. The treatment system according to claim 183, wherein the ischemic disorder includes retinal vein occlusion, and wherein the transducer is configured to apply the energy so as to treat the retinal vein occlusion.

186. The treatment system according to claim 183, wherein the ischemic disorder includes a chronic ischemic disorder of the subject, and wherein the transducer is configured to apply the energy so as to treat the chronic ischemic disorder.

187. The treatment system according to claim 183, wherein the ischemic disorder includes an acute ischemic event of the subject, and wherein the transducer is configured to apply the energy so as to treat the acute ischemic event.

188. The treatment system according to claim 187, wherein the acute ischemic event includes acute ischemic stroke of the subject, and wherein the transducer is configured to apply the energy so as to treat the acute ischemic stroke.

189. A treatment system for treating a central nervous system (CNS) disorder of a subject, the treatment system comprising:
a carrier system adapted to be supplied to a body of the subject, the carrier system encapsulating a nitric oxide (NO) facilitator; and
a transducer, adapted to apply ultrasound energy to the carrier system at an energy level sufficient to cause the carrier system to release the NO facilitator in a blood circulation of the subject in a vicinity of a blood-brain barrier (BBB) of the subject and thereby increase clearance of a CNS constituent related to the CNS disorder, from the CNS, through the BBB, and into a systemic blood circulation of the subject, so as to treat the CNS disorder.

190. A diagnostic system for facilitating a diagnosis of a disorder of a central nervous system (CNS) of a subject, the diagnostic system comprising:
a carrier system adapted to be supplied to a body of the subject, the carrier system encapsulating a nitric oxide (NO) facilitator; and
a transducer, adapted to apply ultrasound energy to the carrier system at an energy level sufficient to cause the carrier system to release the NO facilitator in a blood circulation of the subject in a vicinity of a blood-brain barrier (BBB) of the subject and thereby increase clearance of a CNS constituent related to the CNS disorder, from the CNS, through the BBB, and into another body compartment of the subject, so as to facilitate the diagnosis of the CNS disorder.

191. A treatment system for treating a disorder of a subject, the treatment system comprising:
a carrier system adapted to be supplied to a body of the subject, the carrier system encapsulating a nitric oxide (NO) inhibitor; and
a transducer, adapted to apply ultrasound energy to the carrier system at an energy level sufficient to cause the carrier system to release the NO inhibitor in a blood circulation of the subject in a vicinity of a blood-brain barrier (BBB) of the subject and thereby decrease permeability of the BBB, so as to treat the disorder.

192. A treatment system for treating a disorder of a subject, the treatment system comprising:
a light-activated nitric acid (NO) precursor; and
a light source, adapted to apply light to the NO precursor at a level sufficient to cause the NO precursor to release NO in a vicinity of a blood-brain barrier (BBB) of the subject and thereby increase permeability of the BBB, so as to treat the disorder.

193. A treatment system for treating a disorder of a subject, the treatment system comprising:
a carrier system adapted to be supplied to a body of the subject, the carrier system encapsulating a nitric oxide (NO) facilitator; and
a transducer, adapted to apply ultrasound energy to the carrier system at an energy level sufficient to cause the carrier system to release the NO facilitator in a blood circulation of the subject in a vicinity of a blood-brain barrier (BBB) of the subject and thereby increase passage of a substance through the BBB between a spinal cord of the subject and the blood circulation, so as to treat the disorder.

194. A diagnostic system for facilitating a diagnosis of a disorder of a subject, the diagnostic system comprising:
a carrier system adapted to be supplied to a body of the subject, the carrier system encapsulating a nitric oxide (NO) facilitator; and
a transducer, adapted to apply ultrasound energy to the carrier system at an energy level sufficient to cause the carrier system to release the NO facilitator in a blood circulation of the subject in a vicinity of a blood-brain barrier (BBB) of the subject and thereby increase passage of a spinal cord constituent, from a spinal cord of the subject, through the BBB, and into a systemic blood circulation of the subject, so as to facilitate the diagnosis of the disorder.
